(19) United States
(12) Patent Application Publication
(10) Pub. No.: US 2008/0085915 A1
(43) Pub. Date: Apr. 10, 2008

(54) COMPOUNDS AND METHODS FOR THE TREATMENT OF GASTROINTESTINAL AND CENTRAL NERVOUS SYSTEM DISORDERS

(76) Inventors: Cyrus Becker, Fremont, CA (US); Courtney Rubens, San Jose, CA (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/765,554

(22) Filed: Jun. 20, 2007

Related U.S. Application Data
(60) Provisional application No. 60/805,678, filed on Jun. 23, 2006.

(51) Int. Cl. A61K 31/44; A61P 1/00; C07D 453/02

(52) U.S. Cl. 514/305; 546/137

(57) ABSTRACT

The subject invention provides stereoisomeric compounds of formula (X): 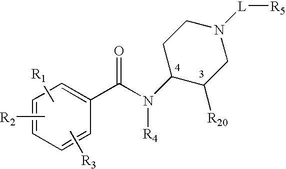 wherein the variables are as defined herein, and compositions for the safe and effective treatment of various gastrointestinal disorders including, but not limited to, gastroparesis, gastroesophageal reflux and related conditions. The compounds of the subject invention are also useful in treating a variety of conditions involving the central nervous system.

### BACKGROUND OF INVENTION

Cisapride is one of a class of compounds known as benzamide derivatives, the parent compound of which is metoclopramide. U.S. Pat. Nos. 4,962,115 and 5,057,525 (collectively “Van Daele” and incorporated by reference in their entireties) disclose N-(3-hydroxy-4-piperidenyl)benzamides of cisapride. Van Daele discloses that these compounds, the pharmaceutically acceptable acid addition salts thereof and the stereochemically isomeric forms thereof, stimulate the motility of the gastrointestinal system.

As a class, these benzamide derivatives have several prominent pharmacological actions. The prominent pharmacological activities of the benzamide derivatives are due to their effects on the neuronal systems which are modulated by the neurotransmitter serotonin. The role of serotonin, and thus the pharmacology of the benzamide derivatives, has been broadly implicated in a variety of conditions for many years. Thus, research has focused on locating the production and storage sites of serotonin as well as the location of serotonin receptors in the human body in order to determine the connection between these sites and various disease states or conditions.

In this regard, it was discovered that a major site of production and storage of serotonin is the enterochromaffin cell of the gastrointestinal mucosa. It was also discovered that serotonin has a powerful stimulating action on intestinal motility by stimulating intestinal smooth muscle, speeding intestinal transit, and decreasing absorption time, as in diarrhea. This stimulating action is also associated with nausea and vomiting.

Because of their modulation of the serotonin neuronal system in the gastrointestinal tract, many of the benzamide derivatives are effective anti-emetic agents and are commonly used to control vomiting during cancer chemotherapy or radiotherapy, especially when highly emetogenic compounds such as cisplatin are used. This action is almost certainly the result of the ability of the compounds to block the actions of serotonin (5HT) at specific sites of action, called the 5HT₃-receptor, which was classically designated in the scientific literature as the serotonin M-receptor. Chemotherapy and radiation therapy may induce nausea and vomiting by the release of serotonin from damaged enterochromaffin cells in the gastrointestinal tract. Release of the neurotransmitter serotonin stimulates both afferent vagal nerve fibers (thus initiating the vomiting reflex) and serotonin receptors in the chemoreceptor trigger zone of the area postrema region of the brain. The anatomical site for this action of the benzamide derivatives, and whether such action is central (CNS), peripheral, or a combination thereof, remains unresolved (Barnes et al., J. Pharm. Pharmacol. 40: 586-588, 1988). Cisapride, like the other benzamide derivatives would appear to be an effective anti-emetic agent based on its ability to modulate the activity of serotonin at the 5HT₃ receptor.

A second prominent action of the benzamide derivatives is in augmenting gastrointestinal smooth muscle activity from the esophagus through the proximal small bowel, thus accelerating esophageal and small intestinal transit as well as facilitating gastric emptying and increasing lower esophageal sphincter tone (Decktor et al., Eur. J. Pharmacol. 147: 313-316, 1988). Although the benzamide derivatives are not cholinergic receptor agonists per se, the aforementioned smooth muscle effects may be blocked by muscarinic receptor blocking agents such as atropine or neuronal transmission inhibitors of the tetrodotoxin type which affect sodium channels. Similar blocking activity has been reported for the contractile effects of serotonin in the small intestine. It is currently believed that the primary smooth muscle effects of the benzamide derivatives are the result of an agonist action upon a new class of serotonin receptors referred to as 5HT₄ receptors which are located on interneurons in the myenteric plexus of the gut wall. Activation of these receptors subsequently enhances the release of acetylcholine from parasympathetic nerve terminals located near surrounding smooth muscle fibers, and it is the combination of acetylcholine with its receptors on smooth muscle membranes which is the actual trigger for muscle contraction.

A discussion of various 5HT receptors, including the 5HT₄ receptor can be found in, for example, U.S. Pat. Nos. 6,331,401 and 6,632,827, which are incorporated by reference herein in their entirety.

Cisapride has been used primarily to treat gastroesophageal reflux disease (GERD). This disease is characterized as the backward flow of the stomach contents into the esophagus. One of the most important factors in the pathogenesis of gastroesophageal reflux disease is a reduction in the pressure barrier due to the failure of the lower esophageal sphincter. Failure of the lower esophageal sphincter can arise due to a low basal pressure, sphincter relaxation, or to a non-compensated increase in intragastric pressure. Other factors in the pathogenesis of the disease are delayed gastric emptying, insufficient esophageal clearing due to impaired peristalsis and the corrosive nature of the reflux material which can damage esophageal mucosa. Cisapride is thought to strengthen the anti-reflux barrier and improve esophageal clearance by increasing the lower esophageal sphincter pressure and enhancing peristaltic contractions.

Because of its activity as a prokinetic agent, cisapride would also appear to be useful to treat dyspepsia, gastroparesis, constipation, post-operative ileus, and intestinal pseudo-obstruction. Dyspepsia is a condition characterized by an impairment of the power or function of digestion that can arise as a symptom of a primary gastrointestinal dysfunction or as a complication due to other disorders such as appendicitis, gallbladder disturbances, or malnutrition. Gastroparesis is a paralysis of the stomach brought about by a motor abnormality in the stomach or as a complication of diseases such as diabetes, progressive systemic sclerosis, anorexia nervosa or myotonic dystrophy. Constipation is a condition characterized by infrequent or difficult evacuation of feces resulting from conditions such as lack of intestinal muscle tone or intestinal spasticity. Post-operative ileus is an obstruction in the intestine due to a disruption in muscle tone following surgery. Intestinal pseudo-obstruction is a condition characterized by constipation, colicky pain, and vomiting, but without evidence of physical obstruction.

Drug toxicity is an important consideration in the treatment of humans and animals. Toxic side effects (adverse effects) resulting from the administration of drugs include a variety of conditions which range from low grade fever to death. Drug therapy is justified only when the benefits of the treatment protocol outweigh the potential risks associated with the treatment. The factors balanced by the practitioner include the qualitative and quantitative impact of the drug to be used as well as the resulting outcome if the drug is not provided to the individual. Other factors considered include the physical condition of the patient, the disease stage and its history of progression, and any known adverse effects associated with a drug.

Drug elimination is typically the result of metabolic activity upon the drug and the subsequent excretion of the drug from the body. Metabolic activity can take place within the vascular supply and/or within cellular compartments or organs. The liver is a principal site of drug metabolism. The metabolic process can be categorized into synthetic and nonsynthetic reactions. In nonsynthetic reactions, the drug is chemically altered by oxidation, reduction, hydrolysis, or any combination of the aforementioned processes. These processes are collectively referred to as Phase I reactions.

In Phase II reactions, also known as synthetic reactions or conjugations, the parent drug, or intermediate metabolites thereof, are combined with endogenous substrates to yield an addition or conjugation product. Metabolites formed in synthetic reactions are, typically, more polar and biologically inactive. As a result, these metabolites are more easily excreted via the kidneys (in urine) or the liver (in bile). Synthetic reactions include glucuronidation, amino acid conjugation, acetylation, sulfoconjugation, and methylation.

More than 90% of a dose of cisapride is metabolized by oxidative N-dealkylation at the piperidine nitrogen or by aromatic hydroxylation occurring on either the 4-fluorophenoxy or benzamide rings.

The administration of cisapride to a human has been found to cause serious adverse effects including CNS disorders, increased systolic pressure, interactions with other drugs, diarrhea, and abdominal cramping. Further, it has been reported that intravenous administration of cisapride demonstrates the occurrence of additional adverse effects not experienced after oral administration of cisapride (Stacher et al. [1987] Digestive Diseases and Sciences 32(11): 1223-1230). It is believed that these adverse effects are caused by the metabolites that result from the oxidative dealkylation or aromatic hydroxylation of the compound which occurs in the cytochrome P450 detoxification system. Cisapride is also subject to a number of undesirable drug/drug interactions that are also a result of metabolism by the cytochrome P450 system.

Between July 1993 and December 1999, cisapride (PROPULSID, Janssen Pharmaceutica Products, L.P.) was reportedly associated with at least 341 serious cardiac arrhythmias. These arrhythmias include ventricular tachycardia, ventricular fibrillation, torsades de pointes, and QT prolongation. Eighty (80) deaths have been reported. As a result of these adverse effects, the product was voluntarily withdrawn from the open market in the United States; however, the drug is available through an investigational limited access program.

The safety of 5HT₄ receptor agonists with gastrointestinal (GI) prokinetic activity has been limited due to cardiac effects (prolongation of QTc intervals, tachycardia, torsades de pointes) and adverse drug interactions due to hepatic cytochrome P-450 metabolism. A GI prokinetic agent of this class that lacks these liabilities would be very valuable in several therapeutic areas including GERD and gastric emptying disorders. Certain cisapride derivatives have been described in U.S. Pat. No. 6,552,046 and WO 01/093849 (incorporated by reference herein in their entireties), however further compounds with even more advantageous properties would be desirable.

It has now been discovered that certain stereoisomers of one such esterified structural and/or functional analog of cisapride have distinct and particularly advantageous properties.

### SUMMARY OF THE INVENTION

The subject invention provides compounds and compositions of formula (X), which are stereoisomeric esterified cisapride analogs, for the safe and effective treatment of various gastrointestinal disorders including, but not limited to, gastroparesis, gastroesophageal reflux and related conditions. The compounds of the subject invention are also useful in treating a variety of conditions involving the central nervous system.

The compounds of the invention comprise compounds of formula X: 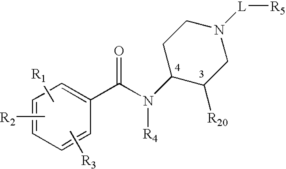
or pharmaceutically acceptable salts thereof, wherein
L, is —(C₁-C₄ alkyl)-NR₉—(C₁-C₄ alkyl)-, —(C₁-C₄ alkyl)-C(O)NR₉—, —(C₁-C₄ alkyl)-, —(C₁-C₄ alkyl)-NR₉C(O)— or —C(O)NR₉—(C₁-C₄ alkyl)-;
R₁ is halogen;
R₂ is amino or mono or di(C₁-C₄ alkyl)amino;
R₃ is C₁-C₄ alkyl, C₁-C₄ alkoxy, or OH;
R₄ is H or C₁-C₄ alkyl;
R₅ is phenyl or naphthyl, each of which is substituted with 1 or 2 groups that are independently C₁-C₄ alkyl, C₁-C₄ alkoxy, OH, —O—C₂-C₄ alkanoyl, halogen, halo C₁-C₄ alkyl, halo C₁-C₄ alkoxy, —CO₂R₁₀, —(C₁-C₄ alkyl)-CO₂R₁₀;
R₉ is H or C₁-C₄ alkyl;
R₁₀ at each occurrence is independently H, C₁-C₄ alkyl optionally substituted with one group that is selected from a 5 or 6 membered monocyclic heterocycloalkyl ring, and OH, quinuclidinyl, —C(O)NH₂, —C(O)NH(C₁-C₄ alkyl), —C(O)NH(C₁-C₄ alkyl) or piperidinyl optionally substituted with C₁-C₄ alkyl; and
R₂₀ is C₁-C₄ alkyl, or C₁-C₄ alkoxy.

The invention also encompasses compositions comprising at least one compound or pharmaceutically acceptable salt of formula (X) and at least one pharmaceutically acceptable excipient, adjuvant, carrier, or solvent.

The compounds of formula (X) are useful in the treatment or prevention of gastroesophageal reflux disease and substantially reduce adverse effects associated with the administration of cisapride. These adverse effects include, but are not limited to, diarrhea, abdominal cramping and elevations of blood pressure and heart rate.

Additionally, the compounds and compositions of the invention are useful in treating emesis and other conditions, including but not limited to dyspepsia, gastroparesis, constipation, post-operative ileus and intestinal pseudo-obstruction. As an added benefit, adverse effects associated with the administration of cisapride are also reduced in these methods of treatment.

Advantageously, the compounds of the subject invention are ligands for the 5HT₄ receptor and, accordingly, can be used to treat conditions mediated through this receptor. These receptors are located in several areas of the central nervous system and the modulation of these receptors can be used to effect desired modulations of the CNS.

Advantageously, the subject invention provides stereoisomeric compounds which contain an ester moiety that does not detract from the ability of these compounds to provide a therapeutic benefit, but which makes them more susceptible to degradation by serum and/or cytosolic esterases, thereby avoiding the cytochrome P450 drug detoxification system associated with adverse effects caused by cisapride and reducing the incidence of such adverse events.

The subject invention further provides methods of treatment comprising the administration of the compounds of formula (X) and therapeutically effective amounts to individuals in need of treatment for gastroesophageal reflux disease, dyspepsia, gastroparesis, constipation, post-operative ileus, and intestinal pseudo-obstruction; and related conditions.

Advantageously, the therapeutic compounds of the subject invention are stable in storage and provide for safer metabolism of the drugs as compared to other drugs; therefore, the compounds of the subject invention can be used with a lower incidence of side effects and toxicity.

In a further aspect, the subject invention pertains to the breakdown products (preferably metabolic breakdown products) which are formed when the therapeutic compounds of the subject invention are acted upon by esterases. These breakdown products can be used as described herein to monitor the clearance of the therapeutic compounds from a patient.

In yet a further aspect, the subject invention provides methods for synthesizing the therapeutic stereoisomeric compounds of the subject invention, as well as intermediates useful in preparing the compounds of interest.

### DETAILED DISCLOSURE

In a further aspect, the invention provides compounds of Formula (X), wherein R₁ is chloro or fluoro.

In another aspect, the invention provides compounds of Formula (X), wherein R₂ is amino.

In still another aspect, the invention provides compounds of Formula (X), wherein R₃ is methoxy, ethoxy, or propoxy.

In yet another aspect, the invention provides compounds of Formula (X), wherein R₄ is H or methyl.

In yet still another aspect, the invention provides compounds of Formula (X−1), i.e., compounds of Formula (X) wherein
R₁ is chloro; R₂ is amino; R₃ is methoxy; R₄ is H, R₂₀ is C₁-C₄ alkoxy, and R₁, R₂, and R₃ have the following orientation on the phenyl ring:

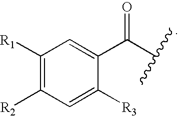

In a further aspect, the invention provides compounds of Formula (X−1), wherein L is —(C₁-C₄ alkyl)-NR₉—(C₁-C₄ alkyl)-, —(C₁-C₄ alkyl)-C(O)NR₉-, or —(C₁-C₄ alkyl)-.

In yet another aspect, the invention provides compounds of Formula (X−2), i.e., compounds of Formula (X−1) having the formula: 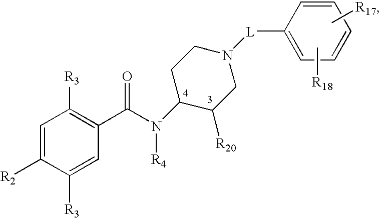
where
R₁₇ is C₁-C₄ alkyl, C₁-C₄ alkoxy, OH, —O—C₂-C₄ alkanoyl, halogen, halo C₁-C₄ alkyl, halo C₁-C₄ alkoxy, —CO₂R₁₀, or —(C₁-C₄ alkyl)-CO₂R₁₀; and
R₁₈ is H, C₁-C₄ alkyl, C₁-C₄ alkoxy, OH, —O—C₂-C₄ alkanoyl, halogen, halo C₁-C₄ alkyl (such as CF₃), halo C₁-C₄ alkoxy (such as OCF₃), —CO₂R₁₀, or —(C₁-C₄ alkyl)-CO₂R₁₀.

In still another aspect, the invention provides compounds of either Formula (X) or Formula (X−2), wherein the bonds at positions 3 and 4 of the piperidinyl ring are cis to each other.

In still another aspect, the orientation of bonds 3 and 4 is as follows:

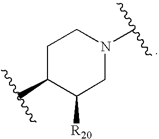

In a preferred aspect, the orientation of bonds 3 and 4 is as follows:

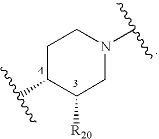

In yet still another aspect, the invention provides compounds of either of Formulas (X) or (X−2), wherein L is —(C₁-C₂ alkyl)-NR₉—(C₁-C₂ alkyl)-; R₁₇ is C₁-C₄ alkyl, C₁-C₄ alkoxy, or halogen; and R₁₈ is H, C₁-C₄ alkyl, C₁-C₄ alkoxy, OH, or —O—C₂-C₄ alkanoyl. Preferably, one of R₁₇ or R₁₈ is at the 4-position of the phenyl group.

In still yet another aspect, the invention provides compounds of either of Formulas (X) or (X−2), wherein L, is —(C₁-C₂ alkyl)-NR₉—(C₁-C₂ alkyl)-; R₁₇ is halogen, halo C₁-C₄ alkyl, or halo C₁-C₄ alkoxy; and R₁₈ is H, C₁-C₄ alkyl, C₁-C₄ alkoxy, OH. Preferably, one of R₁₇ or R₁₈ is at the 4-position of the phenyl group.

In another aspect, the invention provides compounds of either of Formulas (X) or (X-2), wherein L is —(C₁-C₂ alkyl)-NR₉—(C₁-C₂ alkyl)-; R₁₇ is OH, or —O—C₁-C₄ alkanoyl; and R₁₈ is H, C₁-C₄ alkyl, C₁-C₄ alkoxy, or OH. Preferably, one of R₁₇ or R₁₈ is at the 4-position of the phenyl group.

In yet another aspect, the invention provides compounds of either of Formulas (X) or (X−2), wherein L is —(C₁-C₂ alkyl)-NR₉—(C₁-C₂ alkyl)-; R₁₇ is OH, or —O—C₂-C₄ alkanoyl; and R₁₈ is O—C₂-C₄ alkanoyl, halogen, halo C₁-C₄ allyl, or halo C₁-C₄ alkoxy. Preferably, one of R₁₇ or R₁₈ is at the 4-position of the phenyl group.

In still another aspect, the invention provides compounds of either of Formulas (X) or (X−2), wherein L is —(C₁-C₂ alkyl)-NR₉—(C₁-C₂ alkyl)-; R₁₀ is H, C₁-C₄ alkyl optionally substituted with one group that is selected from morpholinyl, pyrrolidinyl, and OH, quinuclidinyl, —C(O)NH₂, or piperidinyl optionally substituted with C₁-C₃ alkyl; R₁₇ is —CO₂R₁₀, or —(C₁-C₄ alkyl)-CO₂R₁₀; and R_{1g} is C₁-C₄ alkyl, C₁-C₄ alkoxy, or OH. Preferably, one of R₁₇ or R₁₈ is at the 4-position of the phenyl group.

In still yet another aspect, the invention provides compounds of either of Formulas (X) or (X−2), wherein L, is —(C₁-C₂ alkyl)-NR₉—(C₁-C₂ alkyl)-; R₁₀ is H, C₁-C₄ alkyl optionally substituted with one group that is selected from morpholinyl, pyrrolidinyl, and OH, quinuclidinyl, —C(O)NH₂, or piperidinyl optionally substituted with C₁-C₃ alkyl; R₁₇ is —CO₂R₁₀; and R₁₈ is C₁-C₄ alkyl (such as methyl), C₁-C₄ alkoxy (such as methoxy), or OH. Preferably, one of R₁₇ or R₁₈ is at the 4-position of the phenyl group.

In still another aspect, the invention provides compounds of either of Formulas (X) or (X−2), wherein L is —(C₁-C₂ alkyl)-NR₉—(C₁-C₂ alkyl)-; R₁₀ is H, C₁-C₄ alkyl optionally substituted with one group that is selected from morpholinyl, pyrrolidinyl, and OH, quinuclidinyl, —C(O)NH₂, or piperidinyl optionally substituted with C₁-C₃ alkyl; R₁₇ is —(C₁-C₄ alkyl)-CO₂R₁₀; and R₁₈ is C₁-C₄ alkyl, C₁-C₄ alkoxy, or OH. Preferably, one of R₁₇ or R₁₈ is at the 4-position of the phenyl group.

In yet another aspect, the invention provides compounds of either of Formulas (X) or (X−2), wherein
L, is —(C₁-C₃ alkyl)-NR₉—(C₁-C₃ alkyl)-;
R₁₀ is H, C₁-C₄ allyl optionally substituted with one group that is selected from a 5 or 6 membered monocyclic heterocycloalkyl ring, and OH, quinuclidinyl, —C(O)NH₂, or piperidinyl optionally substituted with C₁-C₃ alkyl;
R₁₇ is OH, —O—C₂-C₄ alkanoyl, —CO₂R₁₀, or —(C₁-C₄ alkyl)-CO₂R₁₀; and
R₁₈ is H; and
R₂₀ is methoxy or ethoxy (in one aspect, methoxy is preferred.)

In yet still another aspect, the invention provides compounds of either of Formulas (X) or (X−2), wherein
L is —(C₁-C₃ alkyl)-NR₉—(C₁-C₃ alkyl)-;
R₁₀ is H, quinuclidinyl, —C(O)NH₂, or piperidinyl optionally substituted with C₁-C₃ allyl;
R₁₇ is OH, —O—C₂-C₄ alkanoyl, —CO₂R₁₀, or —(C₁-C₄ alkyl)-CO₂R₁₀; and
R₁₈ is H; and
R₂₀ is methoxy or ethoxy (in one aspect, methoxy is preferred.)

In yet still another aspect, the invention provides compounds of either of Formulas (X) or (X−2), wherein L is —(C₁-C₃ alkyl)-NR₉—(C₁-C₃ alkyl)-; R₁₀ is H; R₁₇ is —CO₂R₁₀, or —(C₁-C₄ alkyl)-CO₂R₁₀; and R₁₈ is H.

In still another aspect, the invention provides compounds of either of Formulas (X) or (X−2), wherein L is —(C₁-C₃ alkyl)-NR₉—(C₁-C₃ alkyl)-; R₁₇ is OH, or —O—C₂-C₄ alkanoyl; and R₁₈ is H, methyl, methoxy, OH, F, or Cl.

In yet still another aspect, the invention provides compounds of Formula (X−3), i.e., compounds of Formula (X) or (X−2) having the formula:

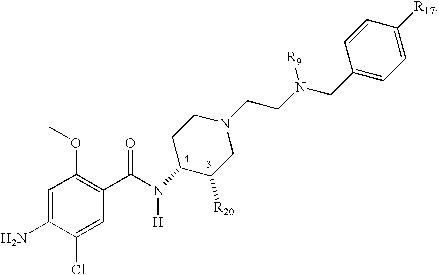

In still yet another aspect, the invention provides compounds of Formula (X−3), wherein R₁₇ is —CO₂R₁₀, or —(C₁-C₄ alkyl)-CO₂R₁₀; R₉ is H or methyl; and R₁₀ is H, C₁-C₄ alkyl optionally substituted with one group that is selected from morpholinyl, pyrrolidinyl, and OH, quinuclidinyl, —C(O)NH₂, or piperidinyl optionally substituted with C₁-C₂ alkyl.

In another aspect, the invention provides compounds of either of Formulas (X) or (X-2), wherein
L is —(C₁-C₃ alkyl)-C(O)NR₉—;
R₁₀ is H, C₁-C₄ alkyl optionally substituted with one group that is selected from a 5 or 6 membered monocyclic heterocycloalkyl ring, and OH, quinuclidinyl, —C(O)NH₂, or piperidinyl optionally substituted with C₁-C₃ alkyl;
R₁₇ is OH, —O—C₂-C₄ alkanoyl, —CO₂R₁₀, or —(C₁-C₄ alkyl)-CO₂R₁₀;
R₁₈ is H; and
R₂₀ is methoxy or ethoxy (in one aspect, methoxy is preferred.)

In yet still another aspect, the invention provides compounds of either of Formulas (X) or (X−2), wherein L is —(C₁-C₂ alkyl)-C(O)NR₉—; R₁₇ is C₁-C₄ alkyl, C₁-C₄ alkoxy, or halogen; and R₁₅ is H, C₁-C₄ alkyl, C₁-C₄ alkoxy, OH, or —O—C₂-C₄ alkanoyl. Preferably, one of R₁₇ or R₁₈ is at the 4-position of the phenyl group.

In still yet another aspect, the invention provides compounds of either of Formulas (X) or (X−2), wherein L, is —(C₁-C₂ alkyl)-C(O)NR₉—; R₁₇ is halogen, halo C₁-C₄ alkyl, or halo C₁-C₄ alkoxy; and R₁₈ is H, C₁-C₄ alkyl, C₁-C₄ alkoxy, OH. Preferably, one of R₁₇ or R₁₈ is at the 4-position of the phenyl group.

In another aspect, the invention provides compounds of either of Formulas (X) or (X−2), wherein L is —(C₁-C₂ alkyl)-C(O)NR₉—; R₁₇ is OH, or —O—C₂-C₄ alkanoyl; and R₁₈ is H, C₁-C₄ alkyl, C₁-C₄ alkoxy, or OH. Preferably, one of R₁₇ or R₁₈ is at the 4-position of the phenyl group.

In yet another aspect, the invention provides compounds of either of Formulas (X) or (X−2), wherein L is —(C₁-C₂ alkyl)-C(O)NR₉—; R₁₇ is OH, or —O—C₂-C₄ alkanoyl; and R₁₈ is O—C₂-C₄ alkanoyl, halogen, halo C₁-C₄ alkyl, or halo C₁-C₄ alkoxy. Preferably, one of R₁₇ or R₁₈ is at the 4-position of the phenyl group.

In still another aspect, the invention provides compounds of either of Formulas (X) or (X−2), wherein L, is —(C₁-C₂ alkyl)-C(O)NR₉—; R₁₀ is H, C₁-C₄ alkyl optionally substituted with one group that is selected from morpholinyl, pyrrolidinyl, and OH, quinuclidinyl, —C(O)NH₂, or piperidinyl optionally substituted with C₁-C₃ alkyl; R₁₇ is —CO₂R₁₀, or —(C₁-C₄ alkyl)-CO₂R₁₀; and R₁₈ is C₁-C₄ alkyl, C₁-C₄ alkoxy, or OH. Preferably, one of R₁₇ or R₁₈ is at the 4-position of the phenyl group.

In still yet another aspect, the invention provides compounds of either of Formulas (X) or (X−2), wherein L, is —(C₁-C₂ alkyl)-C(O)NR₉—; R₁₀ is H, C₁-C₄ alkyl optionally substituted with one group that is selected from morpholinyl, pyrrolidinyl, and OH, quinuclidinyl, —C(O)NH₂, or piperidinyl optionally substituted with C₁-C₃ alkyl; R₁₇ is —CO₂R₁₀; and R₁₈ is C₁-C₄ alkyl (such as methyl), C₁-C₄ alkoxy (such as methoxy), or OH. Preferably, one of R₁₇ or R₁₈ is at the 4-position of the phenyl group.

In still another aspect, the invention provides compounds of either of Formulas (X) or (X−2), wherein L is —(C₁-C₂ allyl)-C(O)NR₉—; R₁₀ is H, C₁-C₄ alkyl optionally substituted with one group that is selected from morpholinyl, pyrrolidinyl, and OH, quinuclidinyl, —C(O)NH₂, or piperidinyl optionally substituted with C₁-C₃ alkyl; R₁₇ is —(C₁-C₄ alkyl)-CO₂R₁₀; and R₁₈ is C₁-C₄ alkyl, C₁-C₄ alkoxy, or OH. Preferably, one of R₁₇ or R₁₈ is at the 4-position of the phenyl group.

In yet another aspect, the invention provides compounds of either of Formulas (X) or (X−2), wherein
L is —(C₁-C₂ alkyl)-C(O)NR₉—;
R₁₀ is H, C₁-C₄ alkyl optionally substituted with one group that is selected from a 5 or 6 membered monocyclic heterocycloalkyl ring, and OH, quinuclidinyl, —C(O)NH₂, or piperidinyl optionally substituted with C₁-C₃ alkyl;
R₁₇ is OH, —O—C₂-C₄ alkanoyl, —CO₂R₁₀, or —(C₁-C₄ allyl)-CO₂R₁₀; and
R₁₈ is H; and
R₂₀ is methoxy or ethoxy (in one aspect, methoxy is preferred.)

In yet still another aspect, the invention provides compounds of either of Formulas (X) or (X−2), wherein
L is —(C₁-C₂ alkyl)-C(O)NR₉—;
R₁₀ is H, quinuclidinyl, —C(O)NH₂, or piperidinyl optionally substituted with C₁-C₃ alkyl;
R₁₇ is OH, —O—C₂-C₄ alkanoyl, —CO₂R₁₀, or —(C₁-C₄ alkyl)-CO₂R₁₀; and
R₁₈ is H; and
R₂₀ is methoxy or ethoxy (in one aspect, methoxy is preferred.)

In yet still another aspect, the invention provides compounds of either of Formulas (X) or (X−2), wherein L, is —(C₁-C₂ allyl)-C(O)NR₉—; R₁₀ is H; R₁₇ is —CO₂R₁₀, or —(C₁-C₄ alkyl)-CO₂R₁₀; and R₁₈ is H.

In still another aspect, the invention provides compounds of either of Formulas (X) or (X−2), wherein L is —(C₁-C₃ allyl)-C(O)NR₉—; R₁₇ is OH, or —O—C₂-C₄ alkanoyl; and R₁₈ is H, methyl, methoxy, OH, F, or Cl.

In yet still another aspect, the invention provides compounds of Formula (X−4), i.e., compounds of Formula (X) or (X−2) having the formula:

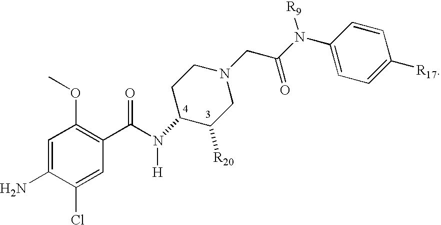

In still yet another aspect, the invention provides compounds of Formula (X−4), wherein R₁₇ is —CO₂R₁₀, or —(C₁-C₄ alkyl)-CO₂R₁₀; R₉ is H or methyl; and R₁₀ is H, C₁-C₄ alkyl optionally substituted with one group that is selected from morpholinyl, pyrrolidinyl, and OH, quinuclidinyl, —C(O)NH₂, or piperidinyl optionally substituted with C₁-C₂ alkyl.

In another aspect, the invention provides compounds of Formula (X−2), wherein
L, is —(C₂-C₄ alkyl)-;
R₁₀ is H, C₁-C₄ alkyl optionally substituted with one group that is selected from a 5 or 6 membered monocyclic heterocycloalkyl ring, and OH, quinuclidinyl, —C(O)NH₂, or piperidinyl optionally substituted with C₁-C₃ alkyl;
R₁₇ is OH, —O—C₂-C₄ alkanoyl, —CO₂R₁₀, or —(C₁-C₄ alkyl)-CO₂R₁₀;
R₁₈ is H; and
R₂₀ is methoxy or ethoxy (in one aspect, methoxy is preferred.)

In yet still another aspect, the invention provides compounds of either of Formulas (X) or (X−2), wherein L, is —(C₂-C₄ allyl)-; R₁₇ is C₁-C₄ alkyl, C₁-C₄ alkoxy, or halogen; and R₁₈ is H, C₁-C₄ alkyl, C₁-C₄ alkoxy, OH, or —O—C₂-C₄ alkanoyl. Preferably, one of R₁₇ or R₁₈ is at the 4-position of the phenyl group.

In still yet another aspect, the invention provides compounds of either of Formulas (X) or (X−2), wherein L, is —(C₂-C₄ alkyl)-; R₁₇ is halogen, halo C₁-C₄ alkyl, or halo C₁-C₄ alkoxy; and R₁₈ is H, C₁-C₄ alkyl, C₁-C₄ alkoxy, OH. Preferably, one of R₁₇ or R₁₈ is at the 4-position of the phenyl group.

In another aspect, the invention provides compounds of either of Formulas (X) or (X−2), wherein L is —(C₂-C₄ alkyl)-; R₁₇ is OH, or —O—C₂-C₄ alkanoyl; and R₁₈ is H, C₁-C₄ alkyl, C₁-C₄ alkoxy, or OH. Preferably, one of R)₇ or R₁₈ is at the 4-position of the phenyl group.

In yet another aspect, the invention provides compounds of either of Formulas (X) or (X−2), wherein L is —(C₂-C₄ alkyl)-; R₁₇ is OH, or —O—C₂-C₄ alkanoyl; and R₁₈ is O—C₂-C₄ alkanoyl, halogen, halo C₁-C₄ alkyl, or halo C₁-C₄ alkoxy. Preferably, one of R₁₇ or R₁₅ is at the 4-position of the phenyl group.

In still another aspect, the invention provides compounds of either of Formulas (X) or (X−2), wherein L, is —(C₂-C₄ alkyl)-; R₁₀ is H, C₁-C₄ alkyl optionally substituted with one group that is selected from morpholinyl, pyrrolidinyl, and OH, quinuclidinyl, —C(O)NH₂, or piperidinyl optionally substituted with C₁-C₃ alkyl; R₁₇ is —CO₂R₁₀, or —(C₁-C₄ alkyl)-CO₂R₁₀; and R₁₈ is C₁-C₄ allyl, C₁-C₄ alkoxy, or OH. Preferably, one of R₁₇ or R₁₈ is at the 4-position of the phenyl group.

In still yet another aspect, the invention provides compounds of either of Formulas (X) or (X−2), wherein L is —(C₂-C₄ alkyl)-; R₁₀ is H, C₁-C₄ alkyl optionally substituted with one group that is selected from morpholinyl, pyrrolidinyl, and OH, quinuclidinyl, —C(O)NH₂, or piperidinyl optionally substituted with C₁-C₃ alkyl; R₁₇ is —CO₂R₁₀; and R₁₈ is C₁-C₄ alkyl (such as methyl), C₁-C₄ alkoxy (such as methoxy), or OH. Preferably, one of R₁₇ or R₁₈ is at the 4-position of the phenyl group.

In still another aspect, the invention provides compounds of either of Formulas (X) or (X−2), wherein L is —(C₂-C₄ alkyl)-; R₁₀ is H, C₁-C₄ alkyl optionally substituted with one group that is selected from morpholinyl, pyrrolidinyl, and OH, quinuclidinyl, —C(O)NH₂, or piperidinyl optionally substituted with C₁-C₃ alkyl; R₁₇ is —(C₁-C₄ alkyl)-CO₂R₁₀; and R₁₈ is C₁-C₄ alkyl, C₁-C₄ alkoxy, or OH. Preferably, one of R₁₇ or R₁₈ is at the 4-position of the phenyl group.

In yet another aspect, the invention provides compounds of either of Formulas (X) or (X−2), wherein
L is —(C₂-C₄ alkyl)-;
R₁₀ is H, C₁-C₄ alkyl optionally substituted with one group that is selected from a 5 or 6 membered monocyclic heterocycloalkyl ring, and OH, quinuclidinyl, —C(O)NH₂, or piperidinyl optionally substituted with C₁-C₃ alkyl;
R₁₇ is OH, —O—C₂-C₄ alkanoyl, —CO₂R₁₀, or —(C₁-C₄ alkyl)-CO₂R₁₀; and
R₁₈ is H; and
R₂₀ is methoxy or ethoxy (in one aspect, methoxy is preferred.)

In yet still another aspect, the invention provides compounds of either of Formulas (X) or (X−2), wherein
L is —(C₂-C₄ alkyl)-;
R₁₀ is H, quinuclidinyl, —C(O)NH₂, or piperidinyl optionally substituted with C₁-C₃ alkyl;
R₁₇ is OH, —O—C₂-C₄ alkanoyl, —CO₂R₁₀, or —(C₁-C₄ alkyl)-CO₂R₁₀; and
R₁₈ is H; and
R₂₀ is methoxy or ethoxy (in one aspect, methoxy is preferred.)

In yet still another aspect, the invention provides compounds of either of Formulas (X) or (X−2), wherein L, is —(C₂-C₄ alkyl)-; R₁₀ is H; R₁₇ is —CO₂R₁₀, or —(C₁-C₄ alkyl)-CO₂R₁₀; and R₁₈ is H.

In still another aspect, the invention provides compounds of either of Formulas (X) or (X−2), wherein L is —(C₂-C₄ alkyl)-; R₁₇ is OH, or —O—C₂-C₄ alkanoyl; and R₁₈ is H, methyl, methoxy, OH, F, or Cl.

In yet still another aspect, the invention provides compounds of Formula (X−5), i.e., compounds of Formula (X) or (X−2) having the formula:

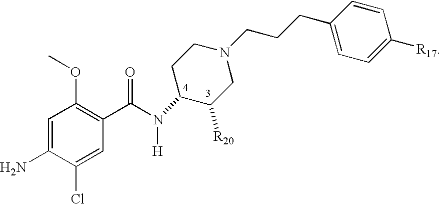

In still yet another aspect, the invention provides compounds of Formula (X−5), wherein R₁₇ is —CO₂R₁₀, or —(C₁-C₄ alkyl)-CO₂R₁₀; R₉ is H or methyl; and R₁₀ is H, C₁-C₄ alkyl optionally substituted with one group that is selected from morpholinyl, pyrrolidinyl, and OH, quinuclidinyl, —C(O)NH₂, or piperidinyl optionally substituted with C₁-C₂ alkyl.

In another aspect, the invention provides compounds of Formula (X-2), wherein
L is —(C₁-C₄ alkyl)-NR₉C(O)—;
R₁₀ is H, C₁-C₄ alkyl optionally substituted with one group that is selected from a 5 or 6 membered monocyclic heterocycloalkyl ring, and OH, quinuclidinyl, —C(O)NH₂, or piperidinyl optionally substituted with C₁-C₃ alkyl;
R₁₇ is OH, —O—C₂-C₄ alkanoyl, —CO₂R₁₀, or —(C₁-C₄ alkyl)-CO₂R₁₀;
R₁₈ is H; and
R₂₀ is methoxy or ethoxy (in one aspect, methoxy is preferred.)

In yet still another aspect, the invention provides compounds of either of Formulas (X) or (X−2), wherein L is —(C₁-C₄ alkyl)-NR₉C(O)—; R₁₇ is C₁-C₄ alkyl, C₁-C₄ alkoxy, or halogen; and R₁₈ is H, C₁-C₄ alkyl, C₁-C₄ alkoxy, OH, or —O—C₂-C₄ alkanoyl. Preferably, one of R₁₇ or R₁₈ is at the 4-position of the phenyl group.

In still yet another aspect, the invention provides compounds of either of Formulas (X) or (X−2), wherein L is —(C₁-C₄ alkyl)-NR₉C(O)—; R)₇ is halogen, halo C₁-C₄ alkyl, or halo C₁-C₄ alkoxy; and R₁₈ is H, C₁-C₄ alkyl, C₁-C₄ alkoxy, OH. Preferably, one of R₁₇ or R₁₈ is at the 4-position of the phenyl group.

In another aspect, the invention provides compounds of either of Formulas (X) or (X−2), wherein L is —(C₁-C₄ alkyl)-NR₉C(O)—; R₁₇ is OH, or —O—C₂-C₄ alkanoyl; and R₂₈ is H, C₁-C₄ alkyl, C₁-C₄ alkoxy, or OH. Preferably, one of R₁₇ or R₁₈ is at the 4-position of the phenyl group.

In yet another aspect, the invention provides compounds of either of Formulas (X) or (X−2), wherein L is —(C₁-C₄ alkyl)-NR₉C(O)—; R₁₇ is OH, or —O—C₂-C₄ alkanoyl; and R₁₈ is O—C₂-C₄ alkanoyl, halogen, halo C₁-C₄ alkyl, or halo C₁-C₄ alkoxy. Preferably, one of R₁₇ or R₁₈ is at the 4-position of the phenyl group.

In still another aspect, the invention provides compounds of either of Formulas (X) or (X−2), wherein L is —(C₁-C₄ alkyl)-NR₉C(O)—; R₁₀ is H, C₁-C₄ alkyl optionally substituted with one group that is selected from morpholinyl, pyrrolidinyl, and OH, quinuclidinyl, —C(O)NH₂, or piperidinyl optionally substituted with C₁-C₃ alkyl; R₁₇ is —CO₂R₁₀, or —(C₁-C₄ alkyl)-CO₂R₁₀; and R₁₈ is C₁-C₄ alkyl, C₁-C₄ alkoxy, or OH. Preferably, one of R₁₇ or R₁₈ is at the 4-position of the phenyl group.

In still yet another aspect, the invention provides compounds of either of Formulas (X) or (X−2), wherein L is —(C₁-C₄ alkyl)-NR₉C(O)—; R₁₀ is H, C₁-C₄ alkyl optionally substituted with one group that is selected from morpholinyl, pyrrolidinyl, and OH, quinuclidinyl, —C(O)NH₂, or piperidinyl optionally substituted with C₁-C₃ alkyl; R₁₇ is —CO₂R₁₀; and R₁₈ is C₁-C₄ alkyl (such as methyl), C₁-C₄ alkoxy (such as methoxy), or OH. Preferably, one of R₁₇ or R₁₈ is at the 4-position of the phenyl group.

In still another aspect, the invention provides compounds of either of Formulas (X) or (X−2), wherein L is —(C₁-C₄ alkyl)-NR₉C(O)—; R₁₀ is H, C₁-C₄ alkyl optionally substituted with one group that is selected from morpholinyl, pyrrolidinyl, and OH, quinuclidinyl, —C(O)NH₂, or piperidinyl optionally substituted with C₁-C₃ alkyl; R₁₇ is —(C₁-C₄ alkyl)-CO₂R₁₀; and R₁₈ is C₁-C₄ alkyl, C₁-C₄ alkoxy, or OH. Preferably, one of R₁₇ or R₁₈ is at the 4-position of the phenyl group.

In yet another aspect, the invention provides compounds of either of Formulas (X) or (X−2), wherein
L is —(C₁-C₄ alkyl)-NR₉C(O)—;
R₁₀ is H, C₁-C₄ alkyl optionally substituted with one group that is selected from a 5 or 6 membered monocyclic heterocycloalkyl ring, and OH, quinuclidinyl, —C(O)NH₂, or piperidinyl optionally substituted with C₁-C₃ alkyl;
R₁₇ is OH, —O—C₂-C₄ alkanoyl, —CO₂R₁₀, or —(C₁-C₄ allyl)-CO₂R₁₀;
R₁₈ is H; and
R₂₀ is methoxy or ethoxy (in one aspect, methoxy is preferred.)

In yet still another aspect, the invention provides compounds of either of Formulas (X) or (X−2), wherein
L is —(C₁-C₄ alkyl)-NR₉C(O)—;
R₁₀ is H, quinuclidinyl, —C(O)NH₂, or piperidinyl optionally substituted with C₁-C₃ alkyl;
R₁₇ is OH, —O—C₂-C₄ alkanoyl, —CO₂R₁₀, or —(C₁-C₄ alkyl)-CO₂R₁₀; and
R₁₈ is H; and
R₂₀ is methoxy or ethoxy (in one aspect, methoxy is preferred.)

In yet still another aspect, the invention provides compounds of either of Formulas (X) or (X−2), wherein L is —(C₁-C₄ alkyl)-NR₉C(O)—; R₁₀ is H; R₁₇ is —CO₂R₁₀, or —(C₁-C₄ alkyl)-CO₂R₁₀; and R₁₈ is H.

In still another aspect, the invention provides compounds of either of Formulas (X) or (X−2), wherein L is —(C₁-C₄ alkyl)-NR₉C(O)—; R₁₇ is OH, or —O—C₂-C₄ alkanoyl; and R₁₈ is H, methyl, methoxy, OH, F, or Cl.

In yet still another aspect, the invention provides compounds of Formula (X−6), i.e., compounds of Formula (X) or (X−2) having the formula:

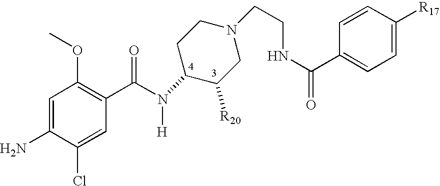

In still yet another aspect, the invention provides compounds of Formula (X−6), wherein R₁₇ is —CO₂R₁₀, or —(C₁-C₄ alkyl)-CO₂R₁₀; R₉ is H or methyl; and R₁₀ is H, C₁-C₄ alkyl optionally substituted with one group that is selected from morpholinyl, pyrrolidinyl, and OH, quinuclidinyl, —C(O)NH₂, or piperidinyl optionally substituted with C₁-C₂ alkyl.

In yet another aspect, the invention provides compounds of Formula (X−6), wherein R₁₇ is —CO₂R₁₀, and R₁₀ is H, or C₁-C₄ allyl optionally substituted with one group that is selected from morpholinyl, pyrrolidinyl. and OH,

In still another aspect, the invention provides compounds of Formula (X-6), wherein R₁₇ is —CO₂R₁₀, and R₁₀ is quinuclidinyl, —C(O)NH₂, or piperidinyl optionally substituted with C₁-C₂ alkyl.

In a further aspect, the invention provides compounds of Formula (X−6), wherein R₁₇ is —CO₂R₁₀, and R₁₀ is H or piperidinyl substituted with C₁-C₂ alkyl.

In another aspect, the invention provides compounds of Formula (XI), i.e., compounds of Formula (X) having the formula: 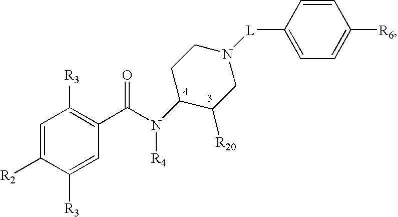
wherein
L is —(C₁-C₄ alkyl)-NR₉—(C₁-C₄ alkyl), —(C₁-C₄ alkyl)-C(O)NR₉, or —(C₁-C₄ alkyl)-;
R₁ is halogen;
R₂ is amino or mono or di(C₁-C₂ alkyl)amino;
R₃ is C₁-C₂ alkoxy;
R₄ is H or methyl;
R₆ is —CO₂R₁₀, —(C₁-C₄ alkyl)-CO₂R₁₀;
R₁₀ is H or C₁-C₄ alkyl substituted with a 5 or 6 membered heterocycloalkyl ring; and
R₂₀ is C₁-C₄ alkoxy.

In another aspect, the invention provides compounds of Formula (XI−1), i.e. compounds of Formula (XI) having the formula: 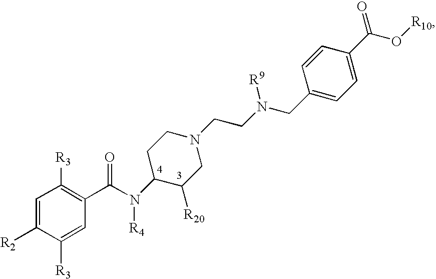
wherein
bonds 3 and 4 of the piperidinyl ring are cis;
R₁ is C₁-C₂ alkoxy;
R₂ is amino or mono or di(C₁-C₂ alkyl)amino;
R₃ is halogen;
R₄ and R₉ are independently H or methyl;
R₁₀ is H, —(C₁-C₃ alkyl)-pyrrolidinyl, —(C₁-C₃ alkyl)-morpholinyl, or —(C₁-C₃ alkyl)-piperidinyl; and R₂₀ is methoxy or ethoxy.

In another aspect, the invention provides compounds of Formula (XI−1) wherein R₁ is methoxy; R₂ is amino; R₃ is chloro; and R₄ is H.

In still another aspect, the invention provides compounds of Formula (XI−1) having the formula: 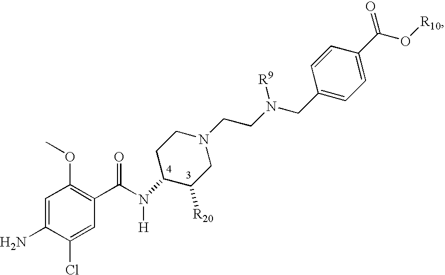
wherein,
R₁₀ is H, —(C₁-C₂ alkyl)-pyrrolidinyl, or —(C₁-C₂ alkyl)-morpholinyl; and R₂₀ is methoxy.

In another aspect, the invention provides compounds of Formula (XI−2), i.e. compounds of Formula (XI) having the formula: 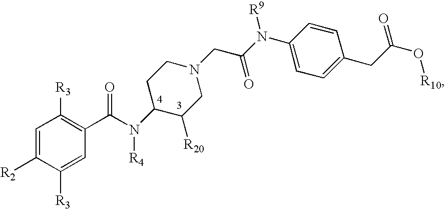
wherein,
bonds 3 and 4 of the piperidinyl ring are cis;
R₁ is C₁-C₂ alkoxy;
R₂ is amino or mono or di(C₁-C₂ alkyl)amino;
R₃ is halogen;
R₄ and R₉ are independently H or methyl;
R₁₀ is H, C₁-C₃ alkyl, or quinuclidinyl; and
R₂₀ is methoxy or ethoxy.

In another aspect, the invention provides compounds of Formula (XI−2) wherein
R₁ is methoxy;
R₂ is amino;
R₃ is chloro;
R₄ is H.

In still another aspect, the invention provides compounds of Formula (XI−2) having the formula: 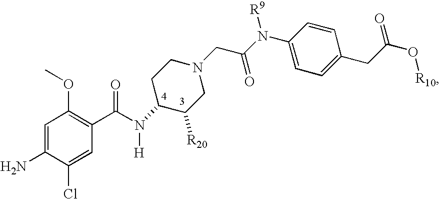
wherein,
R₁₀ is H, C₁-C₂ alkyl, or quinuclidinyl; and
R₂₀ is methoxy.

In another aspect, the invention provides compounds of Formula (XI−3), i.e. compounds of Formula (XI) having the formula: 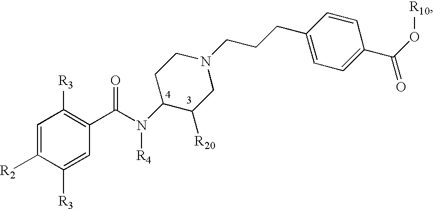
wherein
bonds 3 and 4 of the piperidinyl ring are cis;
R₁ is C₁-C₂ alkoxy;
R₂ is amino or mono or di(C₁-C₂ alkyl)amino;
R₃ is halogen;
R₄ is H or methyl;
R₁₀ is H, or piperidinyl substituted with C₁-C₃ alkyl; and
R₂₀ is methoxy or ethoxy.

In another aspect, the invention provides compounds of Formula (XI−3) wherein
R₁ is methoxy;
R₂ is amino;
R₃ is chloro;
R₄ is H.

In still another aspect, the invention provides compounds of Formula (XI−3) having the formula: 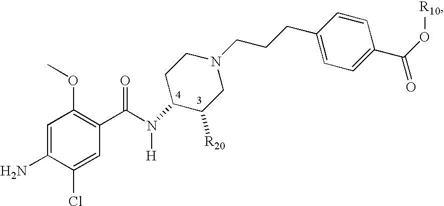
where
R₁₀ is H, or piperidinyl substituted with C₁-C₂ allyl; and
R₂₀ is methoxy.

The invention further provides methods for treating emesis, dyspepsia, gastroparesis, constipation, intestinal pseudo-obstruction, gastroesophageal reflux, or post-operative ileus, the method comprising administering a therapeutically effective amount of a compound or salt according of formula (X) to a patient in need of such treatment.

The subject invention provides compounds that are more susceptible to degradation by serum and/or cytosolic esterases than cisapride, thus avoiding the adverse effects associated with metabolism by cytochrome P450.

Advantageously, the therapeutic compounds of the subject invention are stable in storage but have a relatively short half-life in the physiological environment; therefore, the compounds of the subject invention can be used with a lower incidence of side effects and toxicity.

In a preferred aspect of the subject invention, therapeutic stereoisomeric compounds are provided that are useful in the treatment of gastroesophageal reflux disease and that contain an ester group, which is susceptible to degradation by esterases, thereby breaking down the compound and facilitating its efficient removal from the treated individual. In a preferred aspect, the therapeutic stereoisomeric compounds are metabolized by the Phase I drug detoxification system.

A further aspect of the subject invention pertains to the breakdown products (preferably metabolic breakdown products, i.e., metabolites, generally acids of parent esters) that are produced when the therapeutic compounds of the subject invention are acted upon by an esterase. The presence of these breakdown products in the urine or serum can be used to monitor the rate of clearance of the therapeutic compound from a patient.

Degradation of the compounds of the subject invention by esterases is particularly advantageous for drug metabolism because these enzymes are ubiquitously distributed and their activity is not dependent on age, gender, or disease state to the same extent as oxidative hepatic drug metabolism.

The subject invention further provides methods of treating disorders, such as gastroesophageal reflux disease comprising the administration of a therapeutically effective amount of at least one stereoisomeric structural and/or functional analog of cisapride to an individual in need of treatment. In a specific aspect, the subject invention provides stereoisomeric structural and/or functional analogs of cisapride and pharmaceutical compositions of these esterified compounds.

The subject invention further provides materials and methods for the treatment of emesis and such other conditions, including but not limited to dyspepsia, gastroparesis, constipation, and intestinal pseudo-obstruction, while substantially reducing adverse effects associated with the administration of cisapride.

In a preferred aspect of the subject invention, therapeutic stereoisomeric compounds are provided which are useful in the treatment of gastroesophageal reflux, dyspepsia, gastroparesis, constipation, post-operative ileus, and intestinal pseudo-obstruction and which contain an ester group which is acted upon by esterases thereby breaking down the compound and facilitating its efficient removal from the treated individual.

The subject invention further provides methods of synthesizing the unique and advantageous compounds of the subject invention Particularly, methods of producing and purifying such stereoisomeric compounds are taught. Methods of adding such ester moieties and of producing and purifying stereoisomers, are well known to the skilled artisan and can be readily carried out utilizing the guidance provided herein.

In a preferred aspect, the subject invention pertains to stereoisomerically isolated compounds, and compositions comprising the compounds.

The isolated stereoisomeric forms of the compounds of the invention are substantially free from one another (i.e., in stereoisomeric excess). In other words, the “R.” forms of the compounds are substantially free from the “S” forms of the compounds and are, thus, in stereoisomeric excess of the “S” forms. Conversely, “S” forms of the compounds are substantially free of “R” forms of the compounds and are, thus, in stereoisomeric excess of the “R” forms. In one aspect of the invention, the isolated stereoisomeric compounds are in at least about 80% stereoisomeric excess. In a preferred aspect, the compounds are in at least about 90% stereoisomeric excess. In a more preferred aspect, the compounds are in at least about 95% stereoisomeric excess. In an even more preferred aspect, the compounds are in at least about 97.5% stereoisomeric excess. In a most preferred aspect, the compounds are in at least about 99% stereoisomeric excess. Similarly, the “(+)” and “(−)” forms of the compounds are also provided in stereoisomeric excess.

### DEFINITIONS

As used herein, the term “allyl” includes those alkyl groups of a designed number of carbon atoms. Alkyl groups may be straight, or branched. Examples of “alkyl” include methyl, ethyl, propyl, isopropyl, butyl, iso-, sec- and tert-butyl, pentyl, hexyl, heptyl, 3-ethylbutyl, and the like. If the number of carbon atoms is not specified, the subject “alkyl” moiety has from 1 to 6 carbons.

The term “alkoxy” represents an alkyl group of indicated number of carbon atoms attached to the parent molecular moiety through an oxygen bridge. Examples of alkoxy groups include, for example, methoxy, ethoxy, propoxy and isopropoxy.

By “aryl” is meant an aromatic carbocyclic group having a single ring (e.g., phenyl) that is optionally fused or otherwise attached to other aromatic hydrocarbon rings or non-aromatic hydrocarbon rings. “Aryl” includes multiple condensed rings in which at least one is aromatic, (e.g., 1,2,3,4-tetrahydronaphthyl, naphthyl), wherein each ring is optionally mono-, di-, or trisubstituted with the groups identified below, as well as multiple rings that are not fused, such as, for example, biphenyl or binaphthyl. Preferred aryl groups of the present invention are phenyl, 1-naphthyl, 2-naphthyl, indanyl, indenyl, dihydronaphthyl, fluorenyl, tetralinyl or 6,7,8,9-tetrahydro-5H-benzo[a]cycloheptenyl. More preferred are phenyl, biphenyl, and naphthyl. Most preferred is phenyl. The aryl groups herein are unsubstituted or, as specified, substituted in one or more substitutable positions with various groups. For example, such aryl groups may be optionally substituted with, for example, C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl.

The term “haloalkoxy” refers to an alkoxy group substituted with at least one halogen atom and optionally further substituted with at least one additional halogen atom, where each halogen is independently F, Cl, Br or I. Preferred halogens are F or Cl. Preferred haloalkoxy groups contain 1-6 carbons, more preferably 1-4 carbons, and still more preferably 1-2 carbons. “Haloalkoxy” includes perhaloalkoxy groups, such as OCF₃ or OCF₂CF₃.

The term “heterocycloalkyl” refers to a ring or ring system containing at least one heteroatom that is preferably selected from nitrogen, oxygen, and sulfur, wherein said heteroatom is in a non-aromatic ring. The heterocycloalkyl ring is optionally fused to or otherwise attached to other heterocycloalkyl rings and/or non aromatic hydrocarbon rings and/or phenyl rings. Preferred heterocycloalkyl groups have from 3 to 7 members. More preferred heterocycloalkyl groups have 5 or 6 members. Examples of 5 or 6 membered, monocyclic heterocycloalkyl groups include, for example, morpholinyl, thiomorpholinyl, thiomorpholinyl S-oxide, thiomorpholinyl S,S-dioxide, piperazinyl, pyrrolidinyl, pyrrolinyl, tetrahydropyranyl, piperidinyl, tetrahydrofuranyl, tetrahydrothienyl, oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl S-oxide, tetrahydrothienyl S,S-dioxide and homothiomorpholinyl S-oxide. Preferred heterocycloalkyl groups include piperidinyl, piperazinyl, pyrrolidinyl, thiomorpholinyl, S,S-dioxothiomorpholinyl, morpholinyl, and imidazolidinyl. More preferred are piperidinyl, piperazinyl, pyrrolidinyl, imidazolidinyl, and morpholinyl. The heterocycle groups herein are unsubstituted or, as specified, substituted in one or more substitutable positions with various groups. For example, such heterocycle groups may be optionally substituted with, for example, C₁-C₆ allyl, C₁-C₆ alkoxy, halogen, hydroxy, cyano, nitro, amino, mono(C₁-C₆)alkylamino, di(C₁-C₆)alkylamino, C₂-C₆ alkenyl, C₂-C₆ alkynyl, C₁-C₆ haloalkyl, C₁-C₆ haloalkoxy, amino(C₁-C₆)alkyl, mono(C₁-C₆)alkylamino(C₁-C₆)alkyl, di(C₁-C₆)alkylamino(C₁-C₆)alkyl or ═O.

The term “pharmaceutically acceptable salts” or “a pharmaceutically acceptable salt thereof” refer to salts prepared from pharmaceutically acceptable non-toxic acids or bases including inorganic acids and bases and organic acids and bases. Since the compound of the present invention is basic, salts may be prepared from pharmaceutically acceptable non-toxic acids. Suitable pharmaceutically acceptable acid addition salts for the compound of the present invention include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic, and the like. Preferred acid addition salts are the chloride and sulfate salts. In the most preferred aspect, structural and/or functional analogs of cisapride are administered as the free base or as the mono or dihydrochloride salt.

As used herein, the terms “treatment” and “treating” encompass prophylactic administration of the compound or a pharmaceutical composition comprising the compound (“prophylaxis”) as well as remedial therapy to reduce or eliminate a disease or disorder mentioned herein. Prophylactic administration is intended for prevention of disorders and may be used to treat a subject that is at risk of having or suffering from one or more disorders mentioned herein. Thus, as used herein, the term “treatment”, or a derivative thereof, contemplates partial or complete inhibition of the stated disease state, when an active ingredient of the invention is administered prophylactically or following the onset of the disease state for which such active ingredient of the is administered. “Prophylaxis” refers to administration of the active ingredient(s) to a mammal to protect the mammal from any of the disorders set forth herein, as well as others.

The term “therapeutically effective amount” refers to an amount necessary to achieve a derived therapeutic effect such as: 1) an amount sufficient to alleviate reflux disease, 2) an amount sufficient to alleviate nausea and vomiting, or 3) an amount sufficient to alleviate a condition caused by gastrointestinal motility dysfunction. Therapeutically effective amounts of structural and/or functional analogs of cisapride are encompassed by the above-desclibed dosage amounts and dose frequency schedule.

A “mammal” may be, for example, a mouse, rat, pig, horse, rabbit, goat, cow, cat, dog, or human. In a preferred aspect, the mammal is a human.

The term “individual(s)” is defined as a single mammal to which is administered a compound of the present invention. The mammal may be, for example, a mouse, rat, pig, horse, rabbit, goat, cow, cat, dog, or human. In a preferred aspect, the individual is a human.

The term “esterified cisapride” means therapeutic compounds of the subject invention that are structural and/or functional analogs of cisapride, which contain a hydrolysable group, generally an ester, that does not detract from the ability of these compounds to provide a therapeutic benefit, but which makes these compounds more susceptible to degradation by hydrolases, particularly serum and/or cytosolic esterases, and which reduces the interaction of the cytochrome P-450 drug detoxification system with the cisapride compounds. Esterase-mediated metabolism of esterified cisapride compounds reduces the role of the cytochrome P-450 drug detoxification system in cisapride metabolism and reduces or eliminates adverse effects caused by cisapride.

The term “structural analog” as used herein means that a described compound shares structural characteristics with a parent compound. For example, a structural analog of cisapride may share one or more structural characteristics with the parent cisapride compound, such as a substituted aryl ring connected to a piperidine ring through an amide linker, but differ structurally in other ways, such as the inclusion or deletion of one or more other chemical moieties.

The term “functional analog” as used herein means that a described compound shares a functional characteristic with a parent compound. For example, a functional analog of cisapride may share few, if any, structural characteristics with cisapride, but affect a similar function, for example, 5-HT₄ agonism.

The term “adverse effects” includes, but is not limited to, gastrointestinal disorders such as diarrhea, abdominal cramping, and abdominal grumbling; tiredness; headache; increased systolic pressure; death; ventricular tachycardia; ventricular fibrillation; torsades de pointes; QT prolongation; increased heart rate; neurological and CNS disorders; and interaction of cisapride with other drugs given concurrently such as but not limited to digoxin, diazepam, ethanol, acenocoumarol, cimetidine, ranitidine, paracetamol, and propranolol.

The term “gastroesophageal reflux disease” as used herein means the incidence of, and the symptoms of, those conditions causing the backward flow of the stomach contents into the esophagus.

The terms “eliciting an anti-emetic effect” and “anti-emetic therapy” as used herein mean providing relief from or preventing the symptoms of nausea and vomiting induced spontaneously or associated with emetogenic cancer chemotherapy or irradiation therapy.

The term “treating a condition caused by gastrointestinal motility dysfunction” as used herein means treating the symptoms and conditions associated with this disorder which include, but are not limited to, gastroesophageal reflux disease, dyspepsia, gastroparesis, constipation, post-operative ileus, and intestinal pseudo-obstruction.

The term “prokinetic” as used herein means the enhancement of peristalsis in, and thus the movement through the gastrointestinal tract.

The term “dyspepsia” as used herein means a condition characterized by an impairment of the power or function of digestion that can arise as a symptom of a primary gastrointestinal dysfunction or as a complication due to other disorders such as appendicitis, gallbladder disturbances, or malnutrition.

The term “gastroparesis” as used herein means a paralysis of the stomach brought about by a motor abnormality in the stomach or as a complication of diseases such as diabetes, progressive systemic sclerosis, anorexia nervosa, or myotonic dystrophy.

The term “constipation” as used herein means a condition characterized by infrequent or difficult evacuation of feces resulting from conditions such as lack of intestinal muscle tone or intestinal spasticity.

The term “post-operative ileus” as used herein means an obstruction in the intestine due to a disruption in muscle tone following surgery.

The term “intestinal pseudo-obstructioni” as used herein means a condition characterized by constipation, colicky pain, and vomiting, but without evidence of physical obstruction.

Preparation of Compounds

The chemical synthesis of various analogs of cisapride can be performed by the methods described in European Patent Application No. 0,076,530 A2 published Apr. 13, 1983, U.S. Pat. Nos. 4,962,115 and 5,057,525 and in Van Daele et al., Drug Development Res. 8: 225-232 (1986), the disclosures of which are incorporated herein by reference in their entireties, and modified by the incorporation of an ester group at a point convenient in the synthesis of the disclosed compounds. Exemplary, non-limiting synthesis schemes for certain esterified cisapride analogs of the subject invention are provided in WO 01/093849.

The invention is illustrated further by the following examples, which are not to be construed as limiting the invention in scope or spirit to the specific procedures described in them. Those having skill in the art will recognize that the starting materials may be varied and additional steps employed to produce compounds encompassed by the invention, as demonstrated by the following examples. Those skilled in the art will also recognize that it may be necessary to utilize different solvents or reagents to achieve some of the above transformations. In some cases, protection of reactive functionalities may be necessary to achieve the above transformations. In general, such need for protecting groups, as well as the conditions necessary to attach and remove such groups, will be apparent to those skilled in the art of organic synthesis. When a protecting group is employed, deprotection step may be required. Suitable protecting groups and methodology for protection and deprotection such as those described in Protecting Groups in Organic Synthesis by T. Greene are well known and appreciated in the art.

Unless otherwise specified, all reagents and solvents are of standard commercial grade and are used without further purification. The appropriate atmosphere to run the reaction under, for example, air, nitrogen, hydrogen, argon and the like, will be apparent to those skilled in the art.

### EXAMPLE 1

Preparation of 6-[4R-(4-amino-5-chloro-2-methoxy-benzoylamino)-3S-methoxy-piperidin-1-yl]-hexanoic acid 1-aza-bicyclo[2.2.2]oct-3′R-yl ester, dihydrochloride salt 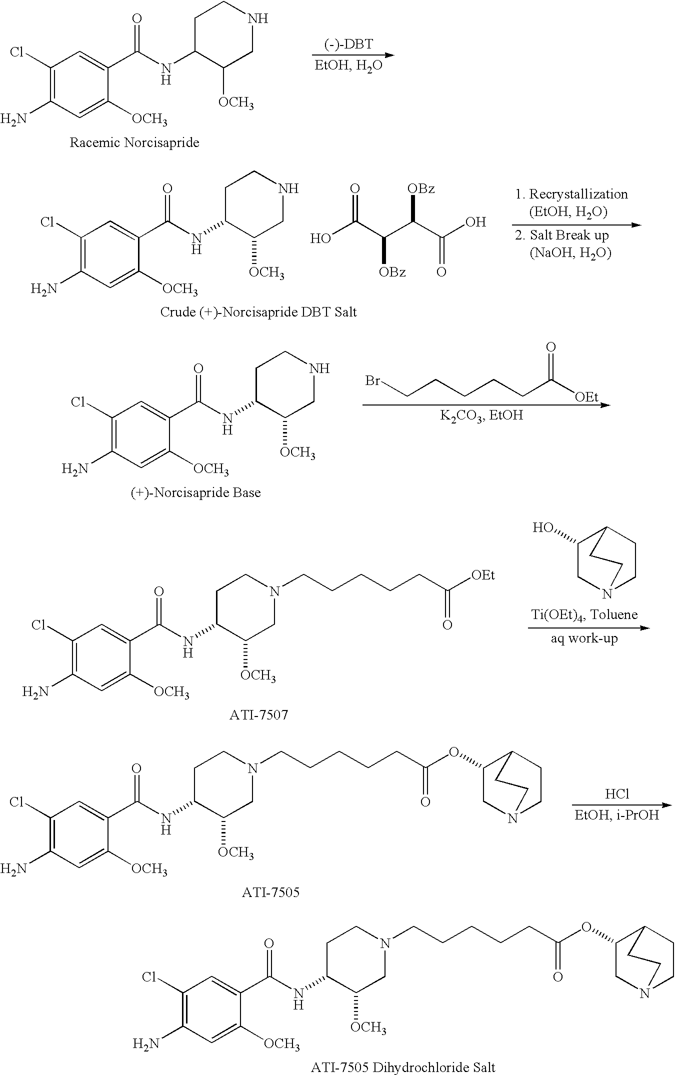
Step 1: Resolution of Racemic Norcisapride

(−)-2,3-Dibenzoyl-L-tartaric acid ((−)-DBT, about 1 part by weight) was dissolved in ethanol and filtered to remove residual particulates. Separately, racemic norcisapride (about 0.8 part by weight) was dissolved in a mixture of ethanol and water and then filtered. The filtrate was heated to about 75° C. before adding the (−)-DBT solution. After stirring at this temperature for about 30 minutes, the mixture was slowly cooled for several hours to about 5° C. and the product salt was collected under vacuum filtration and washed with EtOH/H₂O mixture. The wetcake was recrystallized from EtOH/H₂O by heating to about 79° C. and slow cooling to about 5° C. as before. The product was collected on a vacuum filter and washed with EtOH/H₂O to give a wetcake.

The wetcake was suspended in water and the pH was adjusted to about 12 using 7% (W/W) aq. NaOH. The resulting suspension was stirred for about 3 hours at room temperature before filtering under vacuum and washing the solid material with water and drying under vacuum The product was then retreated with (−)-DBT to form the salt by the same general procedure described above. The isolated salt was then neutralized with aq. NaOH as described above. The product was isolated on a filter and dried as before to provide (+)-norcisapride base (about 0.25 parts by weight). The e.e. by chiral HPLC analysis was about 100% (+)-norcisapride. The optical rotation was about +5° (methanol; 25° C. and 589 nm), confirming the positive isomer of norcisapride.

Step 2: Coupling with Ethyl 6-bromohexanoate

(+)-Norcisapride (about 1 part by weight), potassium carbonate (about 0.48 part by weight) and potassium iodide (about 0.063 part by weight) were suspended in anhydrous USP ethanol. Ethyl 6-bromohexanoate (about 0.76 part by weight) was added slowly to the suspension at room temperature. The mixture was heated to reflux until completion of the reaction. Subsequent cooling to room temperature the reaction mixture was filtered to remove, e.g., inorganic solids, and the filtrate was concentrated under reduced pressure to about one-half the volume. The product was precipitated by slowly adding the crude material to cold water (about 13 parts by weight) with rapid stirring. The precipitate was filtered under vacuum and washed with water and then reprecipitated twice more by dissolution in anhydrous ethanol and slow addition into cold water as before. The resulting wetcake was washed with n-heptane and resuspended in ethyl acetate and n-heptane (1:9; v/v) and stirred for about 1 hour and before filtering and drying under vacuum to yield 0.73 parts by weight of the coupled product as a white solid.

Step 3: Coupling with (R)-3-Quinuclidinol and Dihydrochloride Salt Formation

The ester (1 part by weight) and (R)-3-Quinuclidinol (about 1.12 part by weight) were suspended in toluene before slowly adding titanium (IV) ethoxide (about 0.5 part by weight) to the stirred suspension. The mixture was heated to about 91° C. under a stream of nitrogen, and partial vacuum was applied to the flask through a distillation apparatus in order to azeotropically remove the ethanol. Additional toluene was added as needed to maintain a minimum solvent volume in the flask. The reaction was considered complete after about 33 hours.

The mixture was cooled to about room temperature and extracted five times with water. The organic layer was concentrated under reduced pressure and the resulting residue was redissolved in EtOH/iPrOH (about 1:1 v/v) and then filtered through a 0.45 micron membrane filter to remove any particulates. Concentrated hydrochloric acid was added slowly to the stirred filtrate to precipitate out the desired product as the dihydrochloride salt. The resulting suspension was stirred for several hours at room temperature and collected under vacuum filtration and rinsed with EtOH/iPrOH (1:1; v/v) to provide 0.53 part by weight of the crude product salt.

Crude dihydrochloride salt was resuspended in ethanol and heated to reflux before cooling to room temperature over about 1 hour. The product was collected under vacuum filtration and rinsed with ethanol and then air-dried. The solids were resuspended in ethanol and warmed to about 55° C. to give a clear solution before adding warm isopropanol and the product was allowed to precipitate by slow cooling to room temperature. The resulting suspension was stirred for several hours before vacuum filtering and rinsing with, e.g., isopropanol. The product was vacuum dried, initially at room temperature for several hours and then at about 55° C. until a constant weight was achieved.

### EXAMPLE 2

### Preparation of (R)-quinuclidin-3-yl 6-((3S,4R)-4-(4-amino-2-chloro-6-methoxybenzamido)-3-methoxypiperidin-1-yl)hexanoate

Step 1: Synthesis of ethyl 4-(dibenzylamino)-3-methoxypiperidine-1-carboxylate (1):

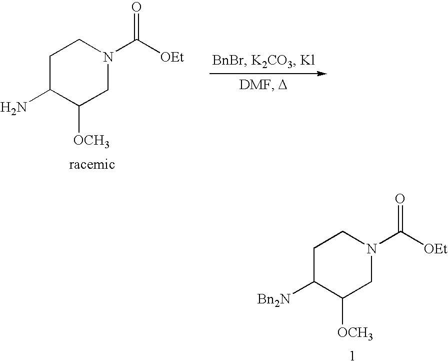

To a solution of racemic ethyl 4-amino-3-methoxypiperidine-1-carboxylate (1 part by mole) in DMF were added benzyl bromide (about 2.2 part by mole), potassium carbonate (about 2.4 part by mole) and potassium iodide (about 0.2 part by mole) respectively. The reaction was heated to about 80° C. (in the specification, delta, or “Δ,” refers to heat). After about 6 hours, the reaction was slowly diluted with water (about 12 pails by volume) and extracted with, for example, ethyl acetate. The organic layer was washed with brine and then dried over anhyh, Na₂SO₄. Subsequent filtration and concentration of the solvent provided the 1 as the yellow-orange oil (1 part by mole).
Step 2. Synthesis of N,N-dibenzyl-3-methoxypiperidin-4-amine (2):

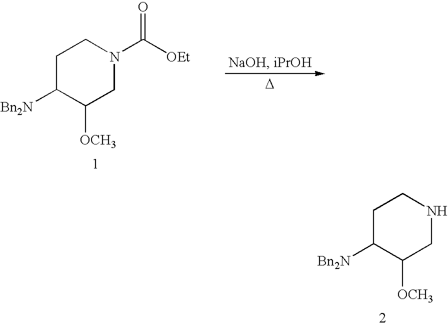

To a solution of 1 was added NaOH (about 10 part by mole) in isopropanol and the mixture was stirred and heated to reflux. After about 3 to about 5 hours, the reaction was cooled to room temperature and the alcoholic solvent was removed via rotary evaporation. The mixture was diluted with water and extracted with ethyl acetate. The organic layer was brined washed before drying over anhyh, Na₂SO₄. Subsequent filtration and concentration of the solvent provided a crude oil which was purified over SiO₂ (CH₂Cl₂:MeOH:NH₄OH; (about) 15:1:0.01) to furnish 2.
Step 3. Synthesis of (3S,4R)-N,N-dibenzyl-3-methoxypiperidin-4-amine (3):

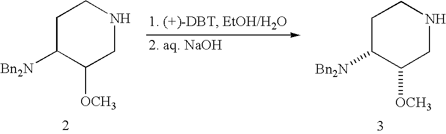

(+)-2,3-Dibenzoyl-D-tartaric acid (about 1.2 part by weight) is dissolved in ethanol before slowly adding to a solution of 2 (about 1 part by weight). The solution is gently warmed and then allowed to cool to room temperature to crystallize the salt product. The salt is filtered and washed with EtOH/H₂O before suspending in water and basifying by adding aq. NaOH (7%, wt/wt) until the pH reaches about 12. The suspension is stirred vigorously at rt and the solid is filtered away, washed with water and vacuum dried to furnish the cis-isomer 3.
Step 4. Synthesis of ethyl 6-((3S,4R)-4-(dibenzylamino)-3-methoxypiperidin-1-yl)hexanoate (4):

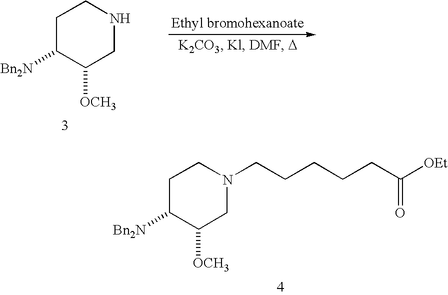

To a solution of 3 (1 part by mole) in DMF are added ethyl bromohexanoate (about 1.2 part by mole), potassium carbonate (about 1.4 part by mole) and potassium iodide (about 0.2 part by mole) respectively. The reaction is then heated to 80° C. After about 8 h, the reaction is slowly diluted with water (about 12 part by volume) and extracted with ethyl acetate. The organic layer is washed with brine and then dried over anhyd. Na₂SO₄. Subsequent filtration and concentration of the solvent furnishes the crude material. Purification over SiO₂ and gives the alkylated material 4,
Step 5. Synthesis of (R)-quinuclidin-3-yl 6-((3S,4R)-4-(dibenzylamino)-3-methoxypiperidin-1-yl)hexanoate (5):

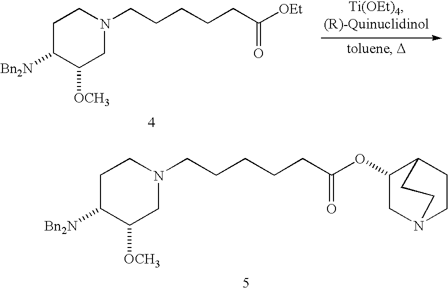

Titanium tetraethoxide is added to a mixture of 4 (1 part by mole) and (R)-(−)-3-quinuclidinol (1 part by mole) in toluene. The reaction mixture is equipped with a dean-stark apparatus before heating to about 90° C. and partial vacuum is then applied (additional toluene is added as needed to main the requisite solvent level). The mixture is then cooled to rt and the reaction is diluted with ethyl acetate and then water is added to the resulting mixture. The organic layer is separated, brine washed, dried over anhyd. Na₂SO₄, filtered and concentrated. Purification over SiO₂ gives the enantiomerically enriched 5.
Step 6. Synthesis of (R)-quinuclidin-3-yl 6-((3S,4R)-4-amino-3-methoxypiperidin-1-yl)hexanoate (6):

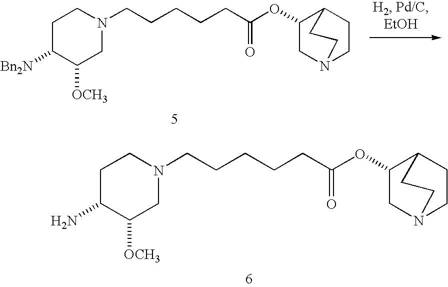

A solution of 5 (1 part by mole) in EtOH is added to a reaction flask containing palladium on carbon (about 0.2 part by mole). The mixture is then evacuated of air before subjecting to hydrogenolysis condition by using atmospheric H₂. Upon completion of the reaction, the palladium is filtered off under a pad of celite followed by EtOH washes. The filtrated is concentrated via rotary evaporation to furnish 6.
Step 7. Synthesis of (R)-quinuclidin-3-yl 6-((3S,4R)-4-(4-amino-2-chloro-6-methoxybenzamido)-3-methoxypiperidin-1-yl)hexanoate (7):

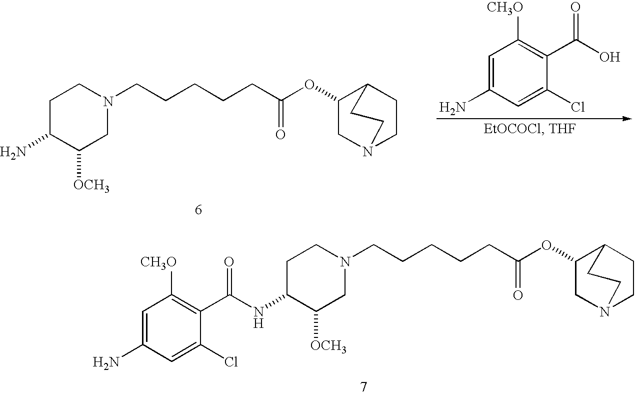

To a solution of, for example, ethyl chloroformate (1 part by mole) in THF at about 0° C. is added the benzoic acid (1 part by mole) in portions. The mixture is warmed to rt for about 1 h before cooling to about 0° C. and adding dropwise a solution of 6 (1 part by mole). The reaction is then warmed to rt. Upon completion of the reaction, reaction is quenched by addition of a sat'd solution of NaHCO₃ and extracting over EA. The organic layer is brine washed, dried over anhyd. Na₂SO₄, filtered and concentrated to furnish the desired product 7.

### EXAMPLE 3

Synthesis of (R)-quinuclidin-3-yl 6-((3S,4R)-4-(4-amino-5-chloro-2-methoxybenzamido)-3-methoxypiperidin-1-yl)hexanoate (or 6-[4R-(4-amino-5-chloro-2-methoxy-benzoylamino)-3S-methoxy-piperidin-1-yl]-hexanoic acid 1-aza-bicyclo[2.2.2]oct-3′R-yl ester):

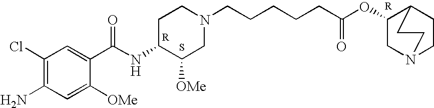

Under acidic conditions, 1-benzylpiperidin-4-one (1) and hydrobromic acid are reacted in the presence of acetic acid to generate N-benzyl-3-bromopiperidin-4-one (2). Treatment of 2 with a sodium methoxide and methanol solution provides 1-benzyl-4,4-dimethoxypiperidin-3-ol (3). [The presence of the beta-amino group negates the possibility of a Favorskii-type reaction.] Methylation of the hydroxyl group is done using a hydride base followed by treatment with iodomethane in the presence of DMF as the solvent to furnish compound 4.

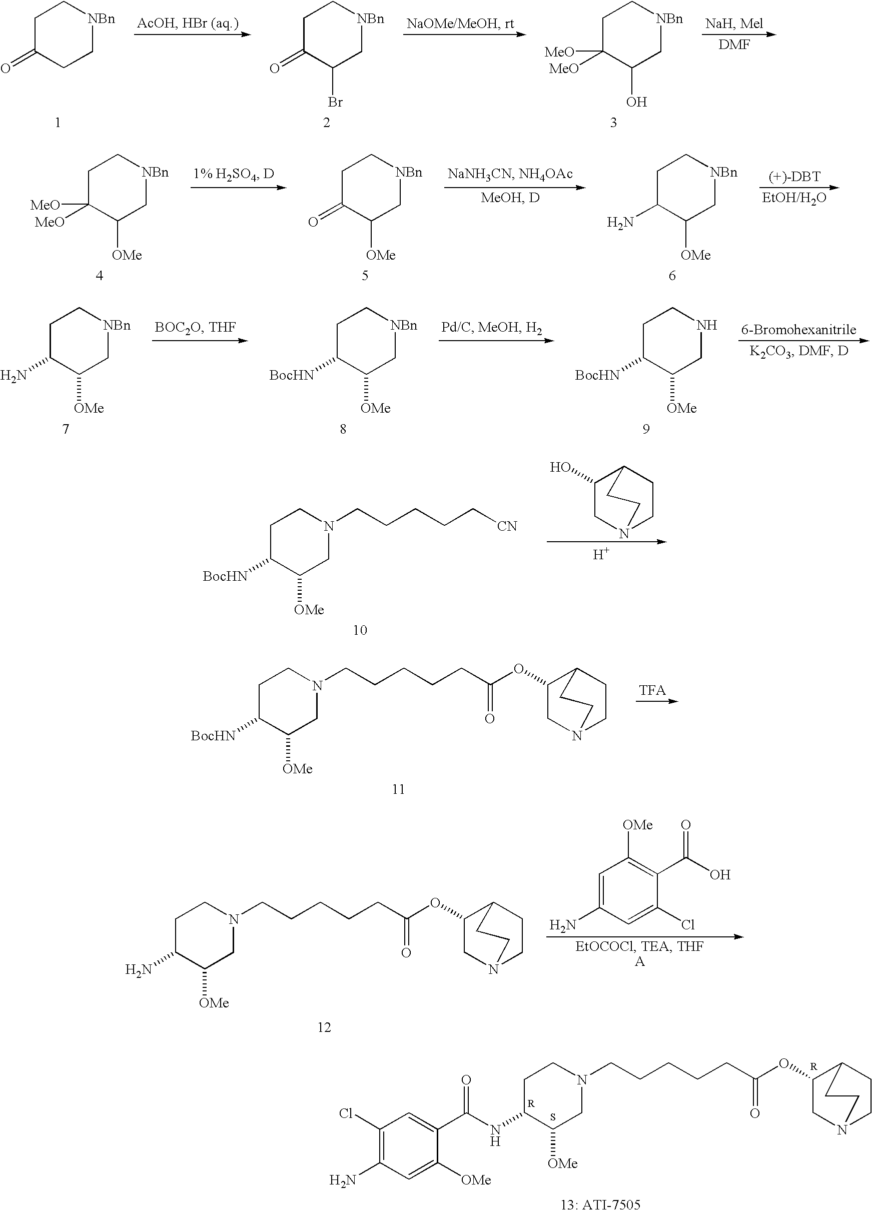

Subsequent acetal hydrolysis using 1% sulfuric acid in the presence of heat yields a piperidine 5, which can then undergo a reductive amination using, for example, sodium cyanoborohydride and ammonium acetate in methanol to yield 1-benzyl-3-methoxypiperidin-4-amine (6). At this stage, 6 can undergo a chiral resolution technique. This can be accomplished, for example, using (+)-DBT or other variant of tartaric acid in the presence of the suitable solvent to afford exclusively asymmetrically pure compound 7. Boc group protection of the primary amine in 7 can be accomplished using Boc anhydride in the presence of THF solvent to obtain 8. A debenzylation reaction by hydrogenolysis using Pd/C in methanol in the presence of atmospheric hydrogen gas set the stage for the alkylation step. Treatment of 6-bromohexanenitrile in the presence of mild base and DMF generates compound 10. A nitrile to ester conversion using (R)-quinuclidinol in the presence of dilute acid generates 11. Subsequent removal of the Boc group using TFA furnishes the free amine, which can undergo a coupling reaction with requisite benzoic acid in the presence of a coupling reagent such as ethyl chloroformate (and more preferably isobutyl chloroformate) to afford ATI-7505 as an enantiomerically pure material.

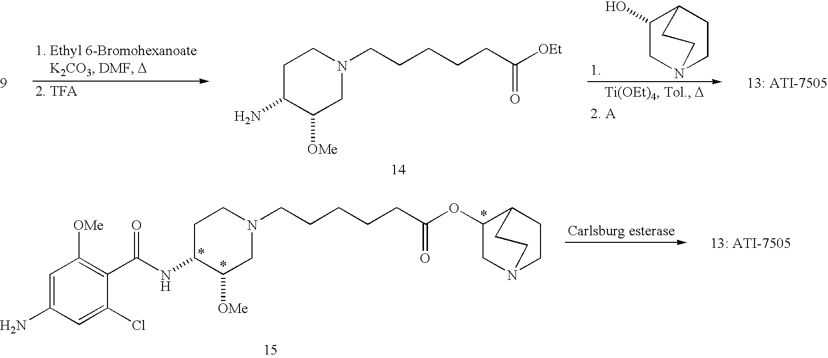

Alternatively, compound 9 can be alkylated using ethyl 6-bromohexanoate in the presence of mild base. Subsequent removal of the Boc group yields compound 14. Titanium mediated transesterification of 14 using (R)-quinuclidinol and titanium tetraethoxide in toluene solvent generates ATI-7505. Carlsburg esterase hydrolyzes esters that are of the S-configuration, therefore leaving intact esters that are of the R configuration. Therefore treatment of diasteriomeric mixtures of 15 with the Carlsburg esterase may also yield ATI-7505.

### EXAMPLE 4

(+) and (−)-norcisapride can be made from its racemic mixture by resolution of the enantiomers using conventional means such as optically resolving acids, according to the method described in U.S. Pat. No. 6,147,093, or in “Enantiomers, Racemates and Resolutions”, by J. Jacques, A. Collet, and S. H. Wilen (Wiley-Interscience, New York, N.Y.), or in S. H. Wilen et al., Tetrahedron (1977) 33:2725.

The 4 isomers can be obtained in low-mg amounts by using preparative column chromatography followed by evaporation of the solvent. This method is useful for preparing small amounts for analytical and characterization purposes. This is a standard separation method used routinely in analytical labs in order to isolate and characterize metabolites.

### EXAMPLE 5

Alternate synthesis of (R)-quinuclidin-3-yl 6-((3S,4R)-4-(4-amino-5-chloro-2-methoxybenzamido)-3-methoxypiperidin-1-yl)hexanoate dihydrochloride salt—ATI-7505 dihydrochloride salt:

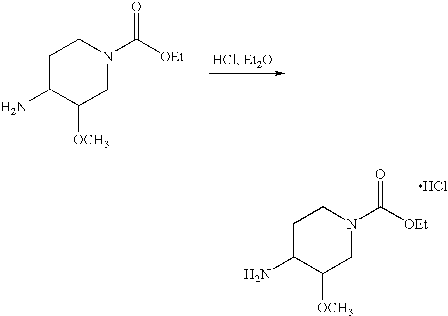

With vigorous stirring hydrogen chloride in diethyl ether (about 1.4 parts by mole) was slowly added to a solution of piperidine carbamate (about 1.0 part by mole). The mixture was allowed to stir for about 8 hours before filtering and washing with diethyl ether. The white solid was further washed with dichloromethane and diethyl ether (about 1.1 ratio by volume) to remove impurities and was subsequently dried under vacuum to obtain the racemic piperidine carbamate hydrochloride salt as a white solid.

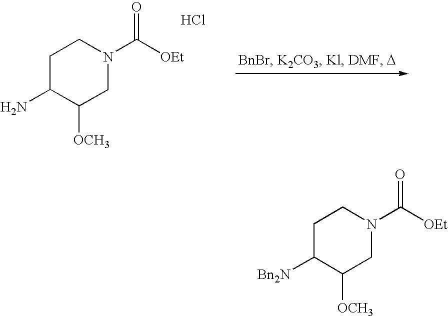

Benzyl bromide (about 2.2 parts by mole) was added to a mixture of the piperidine hydrochloride (about 1.0 parts by mole), potassium carbonate (K₂CO₃, about 2.4 parts by mole), and potassium iodide (KI, about 0.1 parts by mole) in dimethylfoimamide at room temperature (rt). The reaction mixture was heated to about 75° C. After about 18 hours, the reaction was cooled to rt, diluted with water and extracted with ethyl acetate (EA). The organic layer was washed with brine and then dried over anhyd. (anhydrous) sodium sulfate (Na₂SO₄). Subsequent filtration under vacuum and concentration provided the crude oil product. The product was precipitated out by adding a mixture of isopropanol and water (about 1:1 volume ratio) and with stirring. Following vacuum filtration provided a dibenzylamino piperidine as a white solid.

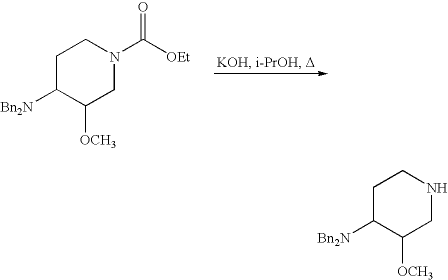

Potassium hydroxide (about 10 parts by mole) was added in portions to a stirred solution of dibenzylamino carbamate (about 1.0 parts by mole) in isopropanol at room temperature and the mixture was stirred and heated to reflux. After about 5 hours the reaction was cooled to room temperature and the solvent was removed under vacuum to approximately half volume. The reaction mixture was diluted with water and extracted with ethyl acetate. Following brine wash, the product was dried over anhydrous Na₂SO₄. Subsequent vacuum filtration provided a piperidine as a semisolid. 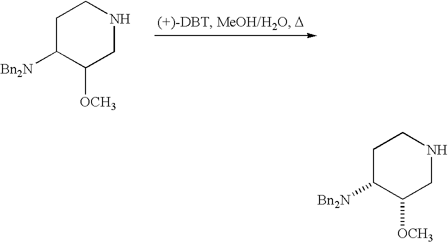
Chiral resolution of 3,4-disubstituted piperidine:

(+)-2,3-Dibenzoyl-D-tartaric acid [(+)-DBT; about 1.0 parts by mole] was dissolved in methanol and was added slowly to a heated solution (about 70° C.) of disubstituted piperidine (about 1.0 parts by mole) in methanol and water (about 1:1 ratio by volume). The mixture was stirred at this temperature for about 1 hour before removing the heat and allowing it to stir at room temperature for several hours, e.g., about 16 hours in one case. The product salt was collected by vacuum filtration and rinsed with methanol and water (about 1:1 ratio by volume). The wet-cake was collected and recrystallized two more times using the same procedure as above.

The wetcake was suspended in water and 1N sodium hydroxide was added to it (to a pH of about 12). The resulting suspension was stirred for about 3 hours at room temperature before extracting with ethyl acetate. The organic layer was washed with brine, filtered and concentrated to provide the enantiomerically enriched 3,4-disubstituted piperidine product as a white solid.

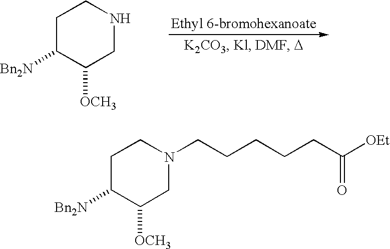

To a mixture of the piperidine (about 1.0 parts by mole), K₂CO₃ (about 1.2 parts by mole) and KI (about 0.1 parts by mole) in DMF solvent was slowly added ethyl 6-bromohexanoate (about 1.1 parts by mole). The reaction was stirred-heat at about 70° C. for about 10 hours before cooling to room temperature and diluting with water and extracting with ethyl acetate. The organic layer was separated and then washed with brined and finally dried over anhydrous Na₂SO₄. Subsequent filtration and concentration provided the crude oil. The crude product was purified via flash column chromatography (e.g., 1:1 ratio of hexanes:ethyl acetate by volume) to provide the product as a light brownish oil.

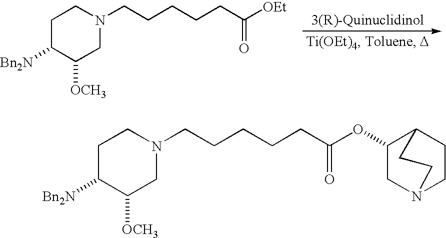

To a mixture of the above piperidine ester (about 1.0 parts by mole) and (3R)-quinuclidinol (about 4.0 parts by mole) was added titanium(IV) tetraethoxide (about 1.0 parts by mole) at room temperature. The reaction was heated to about 85° C. and was run under partial pressure to remove any evolving ethanol. After about 18 hours, the reaction was cooled to rt before diluting with ethyl acetate and quenching with water. The organic layers were then washed with brine and dried over anhydrous Na₂SO₄. Following concentration, the crude oil was purified via flash column chromatography (e.g., about 100:10:1; CH₂Cl₂:MeOH:NH₄OH) to provide the product as a clear oil.

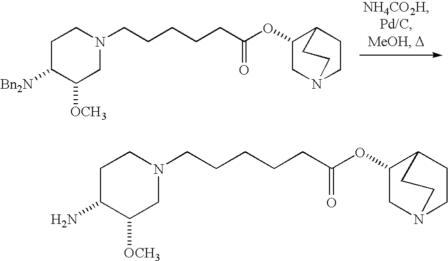

To a reaction flask containing palladium on carbon was added a solution of the above dibenzyl piperidine ester (about 1.0 parts by mole) in methanol, and to this mixture was added ammonium formate (about 4 parts by mole). The reaction was heated to reflux and after about 10 hours, the reaction flask was cooled to room temperature and the palladium on carbon was filtered away, e.g., through a pad of celite. The filtrate was concentrated to an oil, which was purified via flash column chromatography (e.g., SiO₂: about 150:10:1; CH₂Cl₂:CH₃OH: NH₄OH) to provide the amino piperidine ester product as a yellow oil.

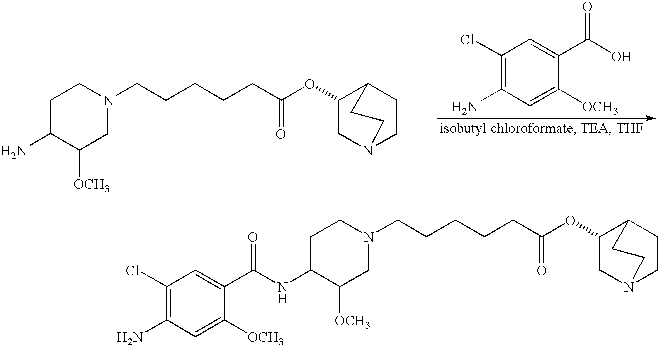

To a stirred solution 4-amino-5-chloro-2-methoxybenzoic acid (about 1.2 parts by mole) and triethylamine (about 2.2 parts by mole) in tetrahydrofuran (THF) was slowly added isobutyl chloroformate (about 1.2 parts by mole) at room temperature. After about 30 minutes, a solution of the piperidine ester (about 1.0 parts by mole) in THF was added to the preformed, mixed anhydride. The reaction was stirred at room temperature for about 14 hours before diluting with a saturated solution of sodium bicarbonate. The product was extracted out using, e.g., ethyl acetate and the separated organic layer was further washed with biine and then dried over anhydrous sodium sulfate. Filtration and concentration provided ATI-7505 free base.

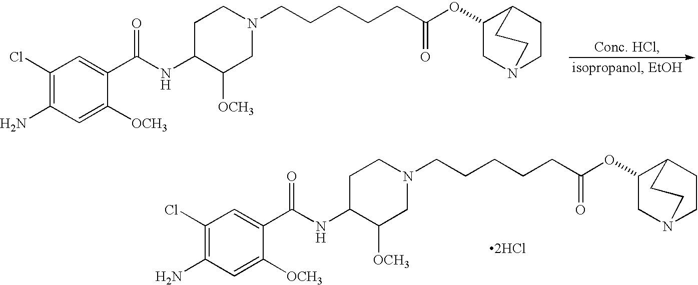

ATI-7505 free-base was dissolved in ethanol and isopropanol (about 1:1 ratio by volume) and cooled in an ice bath. To the ice cold solution was slowly added concentrated hydrochloric acid and then warmed to room temperature. After about 7 hours of stirring at room temperature, the solid was filtered and washed with ethanol and isopropanol (about 1:1 ratio by volume) to provide a wet cake. The wet cake was resuspended in ethanol and then heated to reflux. The stirred solution was warmed to room temperature and allowed to recrystallize. The product was filtered under vacuum rinsed with ethanol and then dried under vacuum to provide ATI-7505 dihydrochloride salt was a white solid.

### EXAMPLE 6

Alternate synthesis of (R)-quinuclidin-3-yl 6-((3S,4R)-4-(4-amino-5-chloro-2-methoxybenzamido)-3-methoxypiperidin-1-yl)hexanoate dihydrochloride salt:

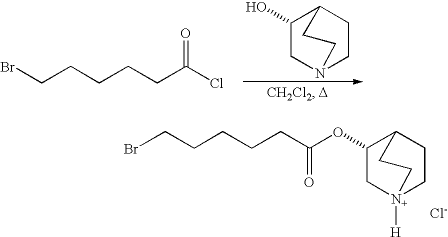

To a mixture of (3R)-quinuclidinol in dichloromethane was added drop-wise 6-bromohexanoyl chloride. The reaction mixture was heated to reflux and after about 18 hours the reaction was cooled to room temperature. The unreacted (3R)-quinuclidinol was filtered away and to the filtrate was added diethyl ether to precipitate out the desired product. The product was filtered under vacuum and washed with CH₂Cl₂ and diethyl ether (about 1:1 ratio by volume) to provide the product as a white solid.

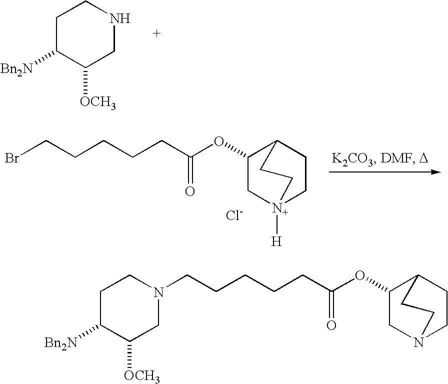

Dibenzylamino piperidine (about 1.0 parts by mole) was added to a mixture of 6-bromoalkanoyl ester (about 1.0 parts by mole) and potassium carbonate (about 2.2 parts by mole) in DMF solvent. The reaction mixture was stirred at about 70° C. for approximately 11 hours before cooling to room temperature and then diluted with a saturated solution of sodium bircarbonate. The product was extracted out with ethyl acetate. Subsequent washing with brine, drying over anhydrous sodium sulfate, filtering and concentrating provided the product as colorless oil.

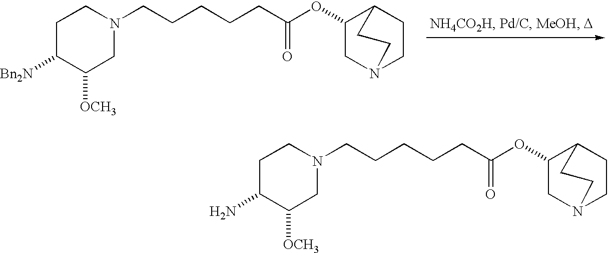

To a reaction flask containing palladium on carbon was added a solution of the above dibenzyl piperidine ester (about 1.0 parts by mole) in methanol, and to this mixture was added ammonium formate (about 4 parts by mole). The reaction was heated to reflux and after about 10 hours, the reaction flask was cooled to room temperature and the palladium on carbon was filtered away, e.g., through a pad of celite. The filtrate was concentrated to an oil, which was purified via flash column chromatography (e.g., SiO₂: about 150:10:1; CH₂Cl₂:CH₃OH: NH₄OH) to provide the amino piperidine ester product as a yellow oil.

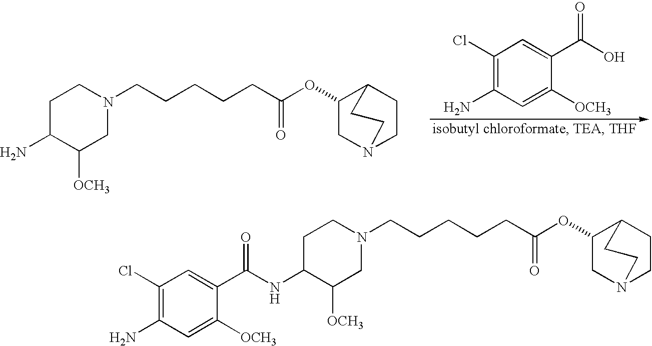

To a stirred solution 4-amino-5-chloro-2-methoxybenzoic acid (about 1.2 parts by mole) and triethylamine (about 2.2 parts by mole) in tetrahydrofuran (THF) was slowly added isobutyl chloroformate (about 1.2 parts by mole) at room temperature. After about 30 minutes, a solution of the piperidine ester (about 1.0 parts by mole) in THF was added to the preformed, mixed anhydride. The reaction was stirred at room temperature for about 14 hours before diluting with a saturated solution of sodium bicarbonate. The product was extracted out using, e.g., ethyl acetate and the separated organic layer was further washed with brine and then dried over anhydrous sodium sulfate. Filtration and concentration provided ATI-7505 free base.

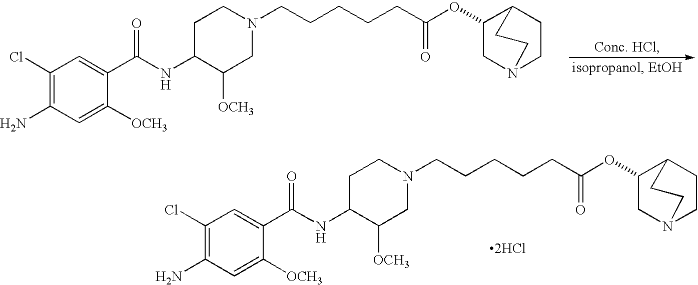

ATI-7505 free-base was dissolved in ethanol and isopropanol (about 1:1 ratio by volume) and cooled in an ice bath. To the ice cold solution was slowly added concentrated hydrochloric acid and then warmed to room temperature. After about 7 hours of stirring at room temperature, the solid was filtered and washed with ethanol and isopropanol (about 1:1 ratio by volume) to provide a wet cake. The wet cake was resuspended in ethanol and then heated to reflux. The stirred solution was warmed to room temperature and allowed to recrystallize. The product was filtered under vacuum rinsed with ethanol and then dried under vacuum to provide ATI-7505 dihydrochloride salt was a white solid.

### EXAMPLE 7

Alternate synthesis of (R)-quinuclidin-3-yl 6-((3S,4R)-4-(4-amino-5-chloro-2-methoxybenzamido)-3-methoxypiperidin-1-yl)hexanoate dihydrochloride salt—ATI-7505 dihydrochloride salt:

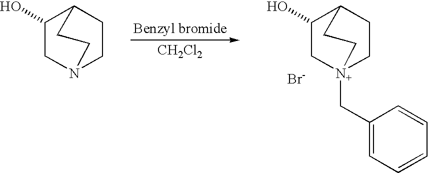

Benzyl bromide (about 1.2 parts by mole) was added to a solution of (3R)-quinuclidinol (about 1.0 parts by mole) in dichloromethane. The reaction was stirred at room temperature for about 4 hours before filtering and rinsing with dichloromethane to provide the product as a white solid.

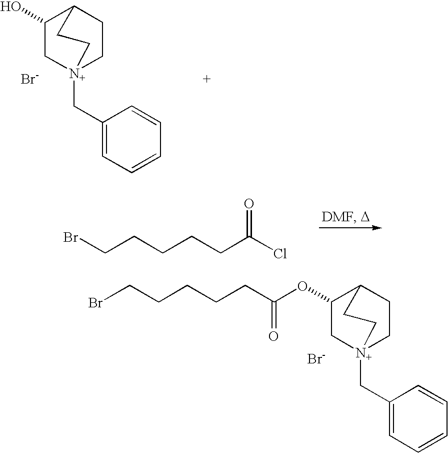

6-Bromohexanoyl chloride (about 1.1 parts by mole) was added to a solution of benzyl protected (3R)-quinuclidinol (about 1.0 parts by mole) and the reaction mixture was heat to about 60° C. After about 12 hours, the reaction was cooled to room temperature and the product was precipitated out by the addition of diethyl ether. Following vacuum filtration and rinsing with ether and drying provided the product as an amorphous solid.

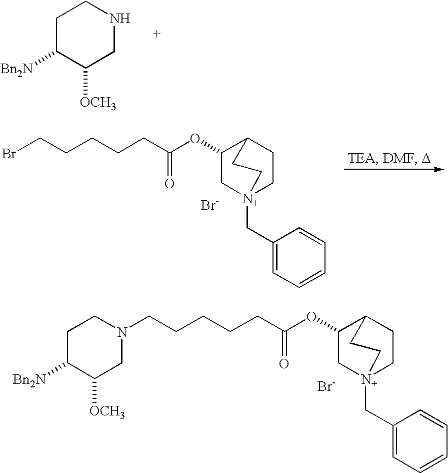

A mixture of the piperidine (about 1.0 parts by mole), the alkanoylhalide ester (about 1.0 parts by mole) and triethylamine (about 2.0 parts by mole) in DMF solvent was heated at about 60° C. for about 6 hours. The reaction was then cooled to room temperature and diluted with a saturated solution of sodium bicarbonate and extracted over ethyl acetate. Following brine wash and drying over anhydrous sodium sulfate the organic layer was concentrated to provide the product as clear oil.

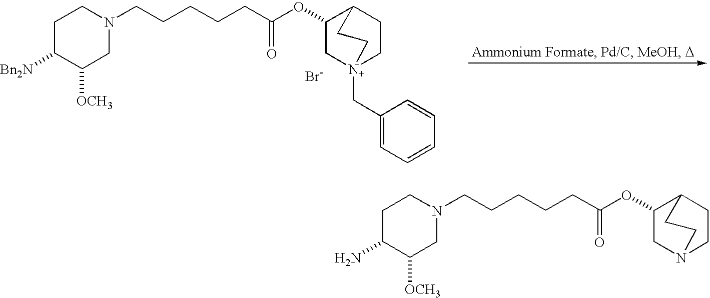

To a reaction flask containing palladium on carbon was added a solution of the dibenzyl piperidine ester (about 1.0 parts by mole) in methanol and to this mixture was added ammonium formate (about 4 parts by mole). The reaction was heated to reflux and after about 10 hours. The reaction flask was then cooled to room temperature and the palladium on carbon was filtered, e.g., through a pad of celite. The filtrate was concentrated to give an oil and was subsequently purified via flash column chromatography (SiO₂: about 150:10:1; CH₂Cl₂: CH₃OH: NH₄OH) to provide the amino piperidine ester product as a yellow oil.

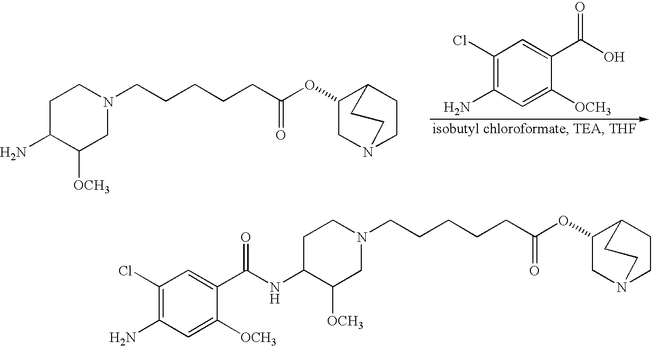

To a stirred solution 4-amino-5-chloro-2-methoxybenzoic acid (about 1.2 parts by mole) and triethylamine (about 2.2 parts by mole) in tetrahydrofuran (THF) was slowly added isobutyl chloroformate (about 1.2 parts by mole) at room temperature. After about 30 minutes, a solution of the piperidine ester (about 1.0 parts by mole) in THF was added to the preformed, mixed anhydride. The reaction was stirred at room temperature for about 14 hours before diluting with a saturated solution of sodium bicarbonate. The product was extracted out using, e.g., ethyl acetate and the separated organic layer was further washed with brine and then dried over anhydrous sodium sulfate. Filtration and concentration provided ATI-7505 free base.

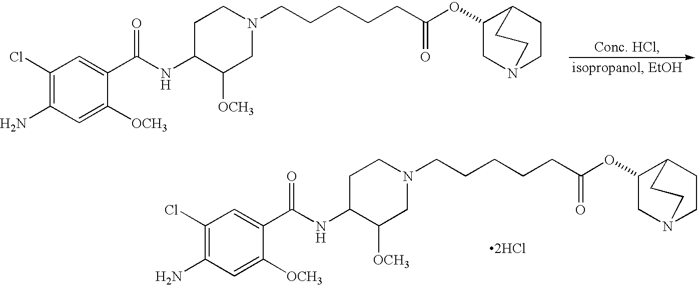

ATI-7505 free-base was dissolved in ethanol and isopropanol (about 1:1 ratio by volume) and cooled in an ice bath. To the ice cold solution was slowly added concentrated hydrochloric acid and then warmed to room temperature. After about 7 hours of stirring at room temperature, the solid was filtered and washed with ethanol and isopropanol (about 1:1 ratio by volume) to provide a wet cake. The wet cake was resuspended in ethanol and then heated to reflux. The stirred solution was warmed to room temperature and allowed to recrystallize. The product was filtered under vacuum rinsed with ethanol and then dried under vacuum to provide ATI-7505 dihydrochloride salt was a white solid.

### EXAMPLE 8

Alternate synthesis of (R)-quinuclidin-3-yl 6-((3S,4R)-4-(4-amino-5-chloro-2-methoxybenzamido)-3-methoxypiperidin-1-yl)hexanoate dihydrochloride salt—ATI-7505 dihydrochloride salt. While specific reaction conditions are recited below for an exemplary synthesis under Method 6, these specifics are not to be construed as limiting the scope of the method. One of skill in the art will recognize that alterations in reaction conditions, including but not limited to reaction times, temperatures and solvents used, may be made under the method Reaction yields, where indicated, are also exemplary and therefore may vary for each run and set of reaction conditions.

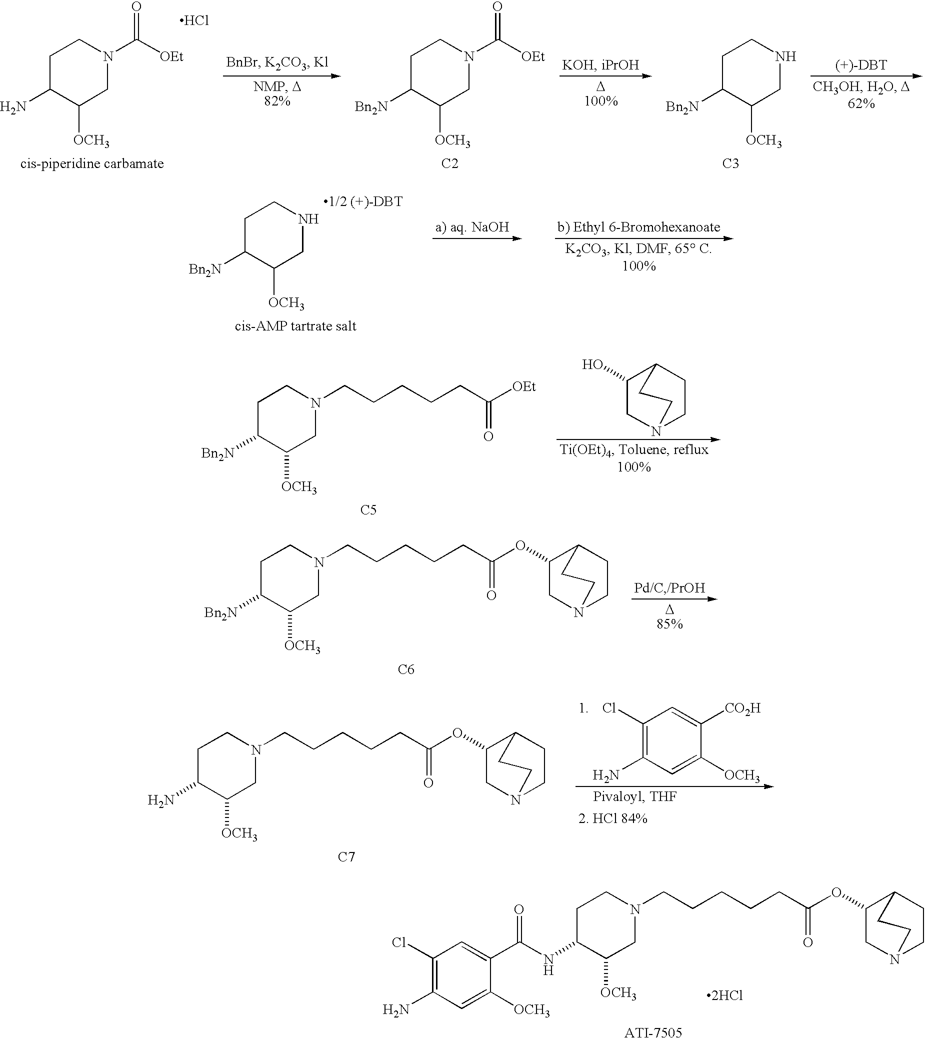

The synthesis of ATI-7505 from cis-AMP tartrate was based on 9.7 g lab run procedure. 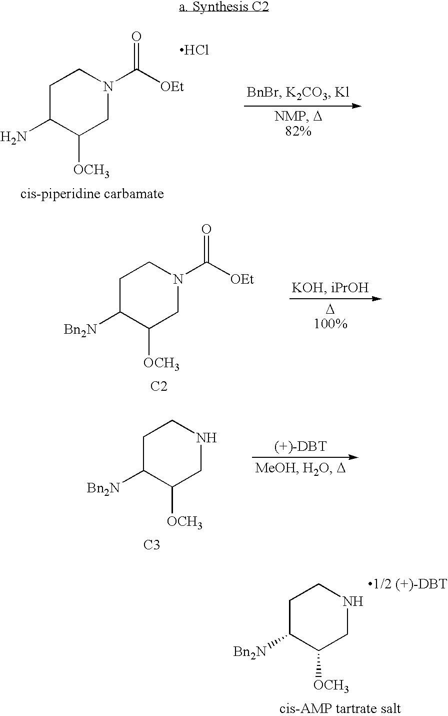
a. Synthesis C2
Raw Materials

cis-piperidine carbamate, 24 Kg

benzyl bromide, 37.8 Kg

KI, 1.67 Kg

K₂CO₃, 48.7 Kg

N-methylpyrrolidone (NMP), 200 Kg

EA (ethyl acetate), 360 Kg

water, 600 kg

isopropyl alcohol (IPA)/water (1:1 w/w), 250 Kg

Procedure

Charge cis-piperidine carbamate (24 Kg, 1 eq.) and K₂CO₃ (48.7 Kg, 6 eq.) to a reactor, followed by adding NMP (200 Kg) to the reactor. Stir the mixture at room temperature for 15 minutes. Add KI (1.67 Kg, 0.1 eq.) to the reactor, followed by adding benzyl bromide (37.8 Kg, 2.2 eq) and increasing the temperature to 75° C. within 60 minutes. Sample the reaction mixture after about 4 hours; the expected reaction time is about 7-9 hours.

After the reaction is deemed completed, add water (350 Kg) to the reactor and extract with EA (120 Kg; 3 times). Collect the EA layer and wash the EA layer with water (200 Kg; 3 times). Concentrate the EA layer to a solid at 70° C. Add IPA/water (1:1, 200 Kg) to the reactor and heat the reactor to about 75-80° C. Add 25 Kg portions of IPA/water (1:1) at 75-80° C. until a clear solution obtained. Slowly cool down the reaction mixture to 5° C. Collect the solid by filtration and dry the wet cake at about 60° C. to obtain C2 (31.7 Kg; 82% yield). HPLC purity for the exemplary batch was 99.3%

b. Synthesis of C3

Raw Material

KOH, 56.3 Kg

IPA, 200 Kg

DCM (dichloromethane), 550 Kg

Water, 1300 Kg

C2, 32 Kg

Procedure

Add C2 (32 Kg, 1 eq), KOH (56.3 Kg, 12 eq) and IPA (200 Kg) to the reactor. Heat the reaction mixture to reflux temperature (about 82° C.). Sample the reaction after about 4 hours; the expected reaction time is about 4-5 hours. After the reaction is completed, remove the IPA by distillation at 50° C. Add DCM (230 Kg) and water (700 Kg) to the reactor and collect both layers. Back extract the water layer with DCM (160 K; 2 times) and combine the DCM layers. Wash the DCM layer with water (200 Kg; 3 times) and concentrate the DCM layer at 70° C. to obtain C3 as an oil and proceed to the next step without isolation (assume a 100% yield).

c. Synthesis of Cis-AMP Tartrate Salt

Raw Materials

Add methanol (260 Kg) and water (130 Kg) to the reactor that contains C3 from the previous step. Add (+)-DBT (15.2 Kg), which has been dissolved in 130 Kg of methanol to the reactor at 70° C., preferably within 60 minutes. Add a portion of methanol (70 Kg) to make ensure a clear solution is obtained before cooling down the reaction mixture to 50° C. The product will come out around 50° C.; slowly cool down to about 10° C. before filtration. Collect solid by filtration and, preferably, check the enantiomeric excess (ee) value and solid content.

Place the solid in a reactor. Add MeOH/water (5:1, 600 Kg) to the reactor and heat the mixture to 70° C. More MeOH/water (5:1) can be added to obtain a clear solution before cooling down the reaction mixture to 50° C. Slowly cool down the mixture to 10° C. before collecting the solid by filtration. Dry the wet cake at about 60° C. In this exemplary run, cis-AMP ½ (+)-DBT (12.6 Kg, 31% weight yield and 62% theoretical yield) was obtained with a HPLC purity 99.8% and an 97.9% ee.
d. Synthesis of the cis-AMP free base 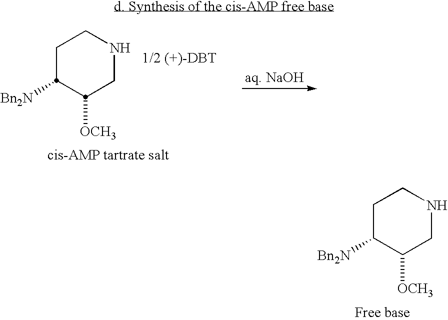
Raw Material

cis AMP ½ DBT, 10 g, 0.0279 mole

isopropyl ether (IPE), 50 ml

water, 50 ml

45% NaOH, 7.2 g, 0.18 mole

Procedure

The piperidine (+)-dibenzoyltartrate salt (10.00 g; 0.0279 mole) was suspended in 30 ml water and 50 ml IPE with aggressive stirring. 45% sodium hydroxide (7.2 g; 0.18 mole) was added dropwise until the solid was dissolved. The IPE layers were washed with water (10 ml; 2 times), and concentration provided a white solid compound of crude free base (7.20 g).
e. Synthesis of C5 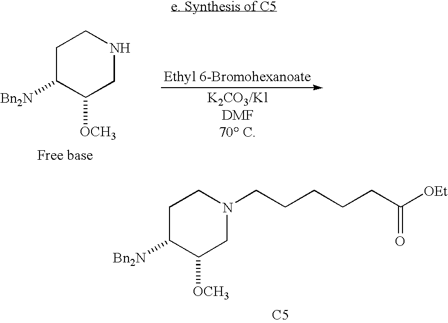
Raw Material

free base, 7.2 g, 0.0232 mole

ethyl 6-bromo-hexanoate, 4.76 g, 0.0214 mole

potassium carbonate, 5.77 g, 0.0418 mole

potassium iodide, 1.39 g, 8.37 mmole

DMF (dimethyl formamide), 30 ml

isopropyl ether (IPE), 50 ml

water, 50 ml.

Procedure

Free base (7.2 g), ethyl 6-bromo-hexanoate (4.75 g; 0.0214 mole), potassium carbonate (5.77 g; 0.0418 mole), potassium iodide (1.39 g; 8.37 mmole), and DMF (30 ml) were charged to a reactor. The reaction mixture was heated to 70° C. for 1 hour and was monitored by HPLC for the completion of the reaction. After 1 hour, the reaction was cooled to room temperature and quenched with 30 ml water and 50 ml IPE. The IPE layers were washed with water (10 ml; 2 times). Concentration provided a yellow oil of crude compound C5 (9.10 g). 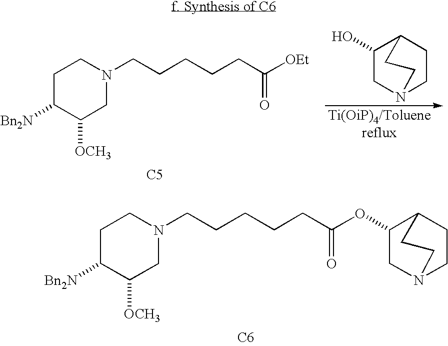
Raw Materials

C5, 9.10 g, 0.0201 mole

(R)-3-quinuclidinol, 5.20 g, 0.0409 mole

Ti(OiP)₄ (titanium (IV) isopropoxide), 1.16 g, 4.08 mmole

toluene, 120 ml

isopropyl ether (IPE), 60 ml

water, 80 ml

Procedure

C5 (9.10 g; 0.0201 mole), (R)-3-quinuclidinol (5.20 g; 0.0409 mole), Ti(OiP)₄ (1.16 g; 4.08 mmole), and toluene (120 ml) were charged in reactor and with stirring. The reaction was equipped with a packing column (24/40; 15 cm in length) and a short path (24/40), which was heated to distill out EtOH, IPA and toluene (oil bath temperature 160° C.). The reaction mixture was monitored by HPLC for the completion of the reaction. In this exemplary synthesis, the HPLC showed the starting material had been completely consumed. Pressure was reduced to facilitate removal of the toluene. The reaction was cooled to room temperature and quenched with 40 ml water and 60 ml IPE, followed by washing the IPE layers with water (20 ml; 2 times) and concentrating, providing a yellow oil compound crude C6 (11.70 g). 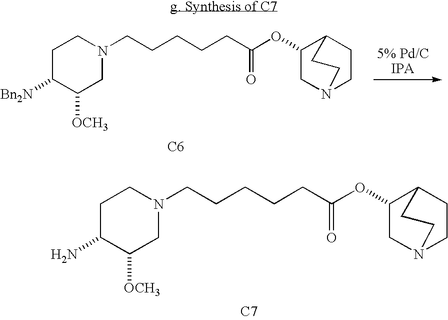
Raw Material

C6, 11.70 g, 0.0220 mole

5% Pd/C, 1.0 g

IPA, 30 ml

Procedure

C7 (11.70 g), 5% Pd/C (1.0 g) and IPA (30 ml) were charged in hydrogenation reactor (N2 inert; H₂ at 5 atmospheres). The mixture was stirred and heated in a 70° C. water bath for 7 hours. The reaction mixture was monitored by HPLC and TLC for the completion of the reaction, which showed that the starting material had been completely consumed. The reaction was cooled to room temperature and filtering through a pad of celite with IPA rinsing. The filtrate was concentrated to provide a 6.66 g crude oil of C7. 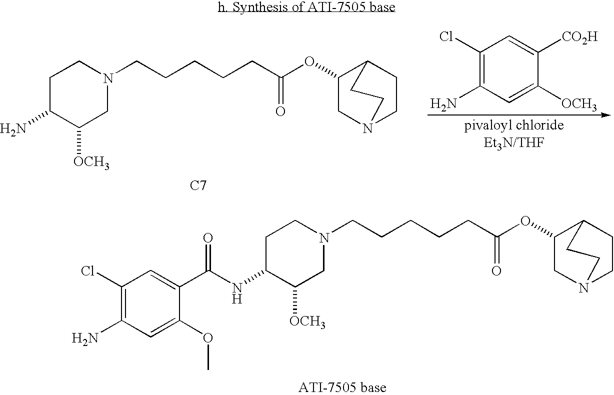
Raw Material

4-amino-5-chloro-2-methoxy-benzoic acid, 5.0 g, 0.0249 mole

THF, 30 g

triethylamine, 4.7 g, 0.0465 mole

pivaloyl chloride, 2.7 g, 0.0225 mole

C7, 6.66 g, 0.0189 mole

diethyl ketone (DEK), 100 ml

32% HCL

water

45% NaOH

Procedure

Pivaloyl chloride (2.7 g; 0.0225 mol) was added dropwise to a solution of the 4-amino-5-chloro-2-methoxy-benzoic acid (5.0 g; 0.0249 mol) and triethylamine (4.7 g; 0.0465 mmol) in THF (20 g) at room temperature. The reaction turned cloudy upon addition and after 60 minutes to this preformed mixed anhydride was added a solution of C7 (6.66 g; 0.0189 mol) in THF (10 g) and allowed to stir at room temperature. HPLC and TLC showed the starting material had been completely consumed.

The reaction was quenched with water (40 ml) and DEK (40 ml), and 32% HCl was added to pH=4.0. The combined organic layers were washed with water (10 ml; 2 times) and the aqueous layer was collected. DEK (60 ml) was added to the aqueous layer and 45% NaOH was added to pH=12. Extract, separate and drain off the aqueous layer. The combined organic layers were washed with water (10 ml; 2 times) and concentrated to provide 12.01 g yellow oil ATI-7505 base. 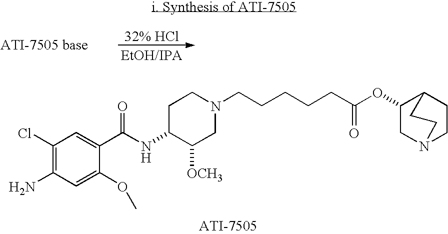
Raw Material

ATI-7505 base, 12.01 g

Ethanol (EtOH), 50 ml

IPA, 70 ml

32% HCL

Procedure

The 12.01 g crude product of ATI-7505 base was dissolved in 50 ml EtOH. Concentrated 32% HCl was added slowly with stirring to pH=4.1. After stirring about 16 hours, 50 ml IPA was added and the reaction was stirred for 2 hours. The solid was filtered and rinsed with 20 ml IPA. The solid was dried to constant weight to provide 9.71 g of ATI-7505 as a white solid. HPLC purity was 98.65%.

The following compounds are prepared essentially according to methods and procedures described above. The compound names were generated using either ChemDraw Ultra v. 8 or later, which is available from Cambridgesoft Corporation or ACD Namepro software, version 6.0.

| Cmpnd. | | MW (Mass |
|---|---|---|
| No. | ChemDraw Chemical Name | Spec) |
| 1 | benzyl 3-((3S,4R)-4-(4-amino-5-chloro-2- | 475.97 |
| | methoxybenzamido)-3-methoxypiperidin-1-yl)propanoate | (calculated) |
| 2 | isopropyl 3-((3R,4S)-4-(4-amino-5-chloro-2- | 428 (MH+) |
| | methoxybenzamido)-3-methoxypiperidin-1-yl)propanoate | 450 (MNa+) |
| 3 | 4-(methylsulfonyl)benzyl 3-((3R,4S)-4-(4-amino-5- | 554 (MH+) |
| | chloro-2-methoxybenzamido)-3-methoxypiperidin-1- | 576 (MNa+) |
| | yl)propanoate | |
| 4 | (tetrahydro-2H-pyran-2-yl)methyl 3-((3R,4S)-4-(4-amino- | 484 (MH+) |
| | 5-chloro-2-methoxybenzamido)-3-methoxypiperidin-1- | 506 (MNa+) |
| | yl)propanoate | |
| 5 | cyclohexyl 3-((3R,4S)-4-(4-amino-5-chloro-2- | 468 (MH+) |
| | methoxybenzamido)-3-methoxypiperidin-1-yl)propanoate | |
| 6 | neopentyl 3-((3S,4R)-4-(4-amino-5-chloro-2- | 456 (MH+) |
| | methoxybenzamido)-3-methoxypiperidin-1-yl)propanoate | 478 (MNa+) |
| 7 | 4-methoxybenzyl 3-((3R,4S)-4-(4-amino-5-chloro-2- | 505 (MH+) |
| | methoxybenzamido)-3-methoxypiperidin-1-yl)propanoate | 528 (MNa+) |
| 8 | pyridin-4-ylmethyl 3-((3R,4S)-4-(4-amino-5-chloro-2- | 477 (MH+) |
| | methoxybenzamido)-3-methoxypiperidin-1-yl)propanoate | |
| 9 | 2-((3S,4R)-4-(4-amino-5-chloro-2-methoxybenzamido)-3- | 394 (MNa+) |
| | methoxypiperidin-1-yl)acetic acid | |
| 10 | 2-(pyrrolidin-1-yl)ethyl 4-(((2-((3S,4R)-4-(4-amino-5- | 602 (MH+) |
| | chloro-2-methoxybenzamido)-3-methoxypiperidin-1- | 600 (MH−) |
| | yl)ethyl)(methyl)amino)methyl)benzoate | |
| 11 | 1-methylpiperidin-4-yl 4-(((2-((3S,4R)-4-(4-amino-5- | 602 (MH+) |
| | chloro-2-methoxybenzamido)-3-methoxypiperidin-1- | 600 (MH−) |
| | yl)ethyl)(methyl)amino)methyl)benzoate | |
| 12 | 2-morpholinoethyl 4-(((2-((3S,4R)-4-(4-amino-5-chloro- | 618 (MH+) |
| | 2-methoxybenzamido)-3-methoxypiperidin-1- | 616 (MH−) |
| | yl)ethyl)(methyl)amino)methyl)benzoate | |
| 13 | 4-fluorobenzyl 2-((3S,4R)-4-(4-amino-5-chloro-2- | 480 (MH+) |
| | methoxybenzamido)-3-methoxypiperidin-1-yl)acetate | |
| 14 | benzyl 2-((3S,4R)-4-(4-amino-5-chloro-2- | 462 (MH+) |
| | methoxybenzamido)-3-methoxypiperidin-1-yl)acetate | 484 (MNa+) |
| 15 | 4-methylbenzyl 2-((3S,4R)-4-(4-amino-5-chloro-2- | 476 (MH+) |
| | methoxybenzamido)-3-methoxypiperidin-1-yl)acetate | 498 (MNa+) |
| 16 | 2-methoxybenzyl 2-((3S,4R)-4-(4-amino-5-chloro-2- | 492 (MH+) |
| | methoxybenzamido)-3-methoxypiperidin-1-yl)acetate | 514 (MNa+) |
| 17 | 4-chlorobenzyl 2-((3S,4R)-4-(4-amino-5-chloro-2- | 496 (MH+) |
| | methoxybenzamido)-3-methoxypiperidin-1-yl)acetate | 518 (MNa+) |
| 18 | 4-methoxybenzyl 2-((3R,4R)-4-(4-amino-5-chloro-2- | 492 (MH+) |
| | methoxybenzamido)-3-methoxypiperidin-1-yl)acetate | 514 (MNa+) |
| 19 | piperidin-4-yl 2-((3S,4R)-4-(4-amino-5-chloro-2- | 455 (MH+) |
| | methoxybenzamido)-3-methoxypiperidin-1-yl)acetate | 477 (MNa+) |
| 20 | 2-methoxyethyl 2-((3S,4R)-4-(4-amino-5-chloro-2- | 430 (MH+) |
| | methoxybenzamido)-3-methoxypiperidin-1-yl)acetate | |
| 21 | 2-hydroxyethyl 2-((3S,4R)-4-(4-amino-5-chloro-2- | 416 (MH+) |
| | methoxybenzamido)-3-methoxypiperidin-1-yl)acetate | 438 (MNa+) |
| 22 | 2-chlorobenzyl 2-((3R,4S)-4-(4-amino-5-chloro-2- | 496 (MH+) |
| | methoxybenzamido)-3-methoxypiperidin-1-yl)acetate | 518 (MNa+) |
| 23 | 4-((2-((3S,4R)-4-(4-amino-5-chloro-2- | 491 (MH+) |
| | methoxybenzamido)-3-methoxypiperidin-1- | 489 (MNa+) |
| | yl)ethylamino)methyl)benzoic acid | |
| 24 | 3-hydroxypropyl 4-((2-((3S,4R)-4-(4-amino-5-chloro-2- | 549 (MH+) |
| | methoxybenzamido)-3-methoxypiperidin-1- | 547 (MH−) |
| | yl)ethylamino)methyl)benzoate | |
| 25 | piperidin-4-yl 4-((2-((3S,4R)-4-(4-amino-5-chloro-2- | 574 (MH+) |
| | methoxybenzamido)-3-methoxypiperidin-1- | 596 (MNa+) |
| | yl)ethylamino)methyl)benzoate | 572 (MH−) |
| 26 | 4-(trifluoromethyl)benzyl 2-((3R,4S)-4-(4-amino-5- | 530 (MH+) |
| | chloro-2-methoxybenzamido)-3-methoxypiperidin-1-yl)acetate | 552 (MNa+) |
| 27 | 3-methylbenzyl 2-((3R,4S)-4-(4-amino-5-chloro-2- | 498 (MNa+) |
| | methoxybenzamido)-3-methoxypiperidin-1-yl)acetate | |
| 28 | 3-chlorobenzyl 2-((3R,4S)-4-(4-amino-5-chloro-2- | 496 (MH+) |
| | methoxybenzamido)-3-methoxypiperidin-1-yl)acetate | 518 (MNa+) |
| 29 | 2-(trifluoromethyl)benzyl 2-((3R,4S)-4-(4-amino-5- | 530 (MH+) |
| | chloro-2-methoxybenzamido)-3-methoxypiperidin-1-yl)acetate | 552 (MNa+) |
| 30 | 2-morpholinoethyl 2-((3R,4S)-4-(4-amino-5-chloro-2- | 484 (MH+) |
| | methoxybenzamido)-3-methoxypiperidin-1-yl)acetate | |
| 31 | (tetrahydro-2H-pyran-2-yl)methyl 2-((3R,4S)-4-(4-amino- | 470 (MH+) |
| | 5-chloro-2-methoxybenzamido)-3-methoxypiperidin-1- | 492 (MNa+) |
| | yl)acetate | |
| 32 | 2-fluorobenzyl 2-((3R,4S)-4-(4-amino-5-chloro-2- | 480 (MH+) |
| | methoxybenzamido)-3-methoxypiperidin-1-yl)acetate | 502 (MNa+) |
| 33 | 4-(2-((3S,4R)-4-(4-amino-5-chloro-2- | 505 (MH+) |
| | methoxybenzamido)-3-methoxypiperidin-1- | 503 (MH−) |
| | yl)ethylcarbamoyl)benzoic acid | |
| 34 | piperidin-4-yl 4-(2-((3S,4R)-4-(4-amino-5-chloro-2- | 588 (MH+) |
| | methoxybenzamido)-3-methoxypiperidin-1- | 610 (MNa+) |
| | yl)ethylcarbamoyl)benzoate | |
| 35 | 3-fluorobenzyl 2-((3R,4S)-4-(4-amino-5-chloro-2- | 480 (MH+) |
| | methoxybenzamido)-3-methoxypiperidin-1-yl)acetate | 502 (MNa+) |
| 36 | 3-methoxybenzyl 2-((3R,4S)-4-(4-amino-5-chloro-2- | 492 (MH+) |
| | methoxybenzamido)-3-methoxypiperidin-1-yl)acetate | 514 (MNa+) |
| 37 | 2-(methylsulfonyl)ethyl 2-((3R,4S)-4-(4-amino-5-chloro- | 500 (MNa+) |
| | 2-methoxybenzamido)-3-methoxypiperidin-1-yl)acetate | |
| 38 | isopropyl 2-((3R,4S)-4-(4-amino-5-chloro-2- | 414 (MH+) |
| | methoxybenzamido)-3-methoxypiperidin-1-yl)acetate | 436 (MNa+) |
| 39 | ethyl 2-((3R,4S)-4-(4-amino-5-chloro-2- | 400 (MH+) |
| | methoxybenzamido)-3-methoxypiperidin-1-yl)acetate | 422 (MNa+) |
| 40 | 2-(pyridin-2-yl)ethyl 2-((3R,4S)-4-(4-amino-5-chloro-2- | 477 (MH+) |
| | methoxybenzamido)-3-methoxypiperidin-1-yl)acetate | 499 (MNa+) |
| 41 | pyridin-2-ylmethyl 2-((3R,4S)-4-(4-amino-5-chloro-2- | 485 (MNa+) |
| | methoxybenzamido)-3-methoxypiperidin-1-yl)acetate | |
| 42 | pyridin-3-ylmethyl 2-((3R,4S)-4-(4-amino-5-chloro-2- | 463 (MH+) |
| | methoxybenzamido)-3-methoxypiperidin-1-yl)acetate | 485 (MNa+) |
| 43 | piperidin-3-ylmethyl 2-((3R,4S)-4-(4-amino-5-chloro-2- | 468.97 |
| | methoxybenzamido)-3-methoxypiperidin-1-yl)acetate | (calculated) |
| 44 | cyclohexyl 2-((3R,4S)-4-(4-amino-5-chloro-2- | 454 (MH+) |
| | methoxybenzamido)-3-methoxypiperidin-1-yl)acetate | 476 (MNa+) |
| 45 | 2-(4-(2-((3S,4R)-4-(4-amino-5-chloro-2- | 505 (MH+) |
| | methoxybenzamido)-3-methoxypiperidin-1- | 503 (MH−) |
| | yl)acetamido)phenyl)acetic acid | |
| 46 | ethyl 2-(4-(2-((3S,4R)-4-(4-amino-5-chloro-2- | 533 (MH+) |
| | methoxybenzamido)-3-methoxypiperidin-1- | 531 (MH−) |
| | yl)acetamido)phenyl)acetate | |
| 47 | 1-methylpiperidin-4-yl 2-(4-(2-((3S,4R)-4-(4-amino-5- | 602 (MH+) |
| | chloro-2-methoxybenzamido)-3-methoxypiperidin-1- | 600 (MH−) |
| | yl)acetamido)phenyl)acetate | |
| 48 | 3-hydroxypropyl 2-(4-(2-((3S,4R)-4-(4-amino-5-chloro-2- | 563 (MH+) |
| | methoxybenzamido)-3-methoxypiperidin-1- | 561 (MH−) |
| | yl)acetamido)phenyl)acetate | |
| 49 | quinuclidin-3-yl 2-(4-(2-((3S,4R)-4-(4-amino-5-chloro-2- | 614 (MH+) |
| | methoxybenzamido)-3-methoxypiperidin-1- | 612 (MH−) |
| | yl)acetamido)phenyl)acetate | |
| 50 | 1-methoxypropan-2-yl 2-((3R,4S)-4-(4-amino-5-chloro-2- | 444 (MH+) |
| | methoxybenzamido)-3-methoxypiperidin-1-yl)acetate | 466 (MNa+) |
| 51 | 2,3,4-trimethoxybenzyl 2-((3R,4S)-4-(4-amino-5-chloro- | 552 (MH+) |
| | 2-methoxybenzamido)-3-methoxypiperidin-1-yl)acetate | 574 (MNa+) |
| 52 | 2,3-dimethoxybenzyl 2-((3R,4S)-4-(4-amino-5-chloro-2- | 522 (MH+) |
| | methoxybenzamido)-3-methoxypiperidin-1-yl)acetate | 544 (MNa+) |
| 53 | 1-(4-fluorophenyl)ethyl 2-((3R,4S)-4-(4-amino-5-chloro- | 494 (MH+) |
| | 2-methoxybenzamido)-3-methoxypiperidin-1-yl)acetate | 516 (MNa+) |
| 54 | 3-(4-fluorophenoxy)propyl 2-((3R,4S)-4-(4-amino-5- | 546 (MNa+) |
| | chloro-2-methoxybenzamido)-3-methoxypiperidin-1-yl)acetate | |
| 55 | 3-fluoro-4-methylbenzyl 2-((3S,4R)-4-(4-amino-5-chloro- | 494 (MH+) |
| | 2-methoxybenzamido)-3-methoxypiperidin-1-yl)acetate | |
| 56 | 4-fluoro-3-methylbenzyl 2-((3S,4R)-4-(4-amino-5-chloro- | 494 (MH+) |
| | 2-methoxybenzamido)-3-methoxypiperidin-1-yl)acetate | |
| 57 | 2-fluoro-6-methylbenzyl 2-((3S,4R)-4-(4-amino-5-chloro- | 494 (MH+) |
| | 2-methoxybenzamido)-3-methoxypiperidin-1-yl)acetate | |
| 58 | tetrahydro-2H-pyran-4-yl 2-((3R,4S)-4-(4-amino-5- | 456 (MH+) |
| | chloro-2-methoxybenzamido)-3-methoxypiperidin-1-yl)acetate | |
| 59 | 4-amino-5-chloro-N-((3S,4R)-1-(2-(4- | 463 (MH+) |
| | hydroxyphenylamino)-2-oxoethyl)-3-methoxypiperidin-4-yl)-2- | 461 (MH−) |
| | methoxybenzamide | |
| 60 | 4-(2-((3S,4R)-4-(4-amino-5-chloro-2- | 505 (MH+) |
| | methoxybenzamido)-3-methoxypiperidin-1-yl)acetamido)phenyl | 503 (MH−) |
| | acetate | |
| 61 | 2-(2-methoxyethoxy)ethyl 2-((3R,4S)-4-(4-amino-5- | 474 (MH+) |
| | chloro-2-methoxybenzamido)-3-methoxypiperidin-1-yl)acetate | 496 (MNa+) |
| 62 | 2-(2-(2-methoxyethoxy)ethoxy)ethyl 2-((3R,4S)-4-(4- | 518 (MH+) |
| | amino-5-chloro-2-methoxybenzamido)-3-methoxypiperidin-1- | 540 (MNa+) |
| | yl)acetate | |
| 63 | neopentyl 2-((3S,4R)-4-(4-amino-5-chloro-2- | 442 (MH+) |
| | methoxybenzamido)-3-methoxypiperidin-1-yl)acetate | 464 (MNa+) |
| 64 | 2-(piperazin-2-yl)ethyl 2-((3S,4R)-4-(4-amino-5-chloro-2- | 484 (MH+) |
| | methoxybenzamido)-3-methoxypiperidin-1-yl)acetate | |
| 65 | pyridin-4-ylmethyl 2-((3R,4S)-4-(4-amino-5-chloro-2- | 485 (MNa+) |
| | methoxybenzamido)-3-methoxypiperidin-1-yl)acetate | |
| 66 | 4-(3-((3S,4R)-4-(4-amino-5-chloro-2- | 476 (MH+) |
| | methoxybenzamido)-3-methoxypiperidin-1-yl)propyl)benzoic | 498 (MNa+) |
| | acid | 474 (MH−) |
| 67 | 2-morpholinoethyl 4-(3-((3S,4R)-4-(4-amino-5-chloro-2- | 589 (MH+) |
| | methoxybenzamido)-3-methoxypiperidin-1-yl)propyl)benzoate | 611 (MNa+) |
| | | 587 (MH−) |
| 68 | 2-(pyrrolidin-1-yl)ethyl 4-(3-((3S,4R)-4-(4-amino-5- | 573 (MH+) |
| | chloro-2-methoxybenzamido)-3-methoxypiperidin-1- | 595 (MNa+) |
| | yl)propyl)benzoate | 571 (MH−) |
| 69 | 1-methylpiperidin-4-yl 4-(3-((3S,4R)-4-(4-amino-5- | 573 (MH+) |
| | chloro-2-methoxybenzamido)-3-methoxypiperidin-1- | 595 (MNa+) |
| | yl)propyl)benzoate | 571 (MH−) |
| 70 | (R)-3-((3R,4S)-4-(4-amino-5-chloro-2- | 422 (MNa+) |
| | methoxybenzamido)-3-methoxypiperidin-1-yl)-2- | |
| | methylpropanoic acid | |
| 71 | (R)-methyl 3-((3R,4S)-4-(4-amino-5-chloro-2- | 414 (MH+) |
| | methoxybenzamido)-3-methoxypiperidin-1-yl)-2- | 436 (MNa+) |
| | methylpropanoate | |
| 72 | 4-(methylsulfonyl)benzyl 3-((3R,4S)-4-(4-amino-5- | 568 (MH+) |
| | chloro-2-methoxybenzamido)-3-methoxypiperidin-1-yl)-2- | 590 (MNa+) |
| | methylpropanoate | |
| 73 | 4-fluorobenzyl 3-((3R,4S)-4-(4-amino-5-chloro-2- | 508 (MH+) |
| | methoxybenzamido)-3-methoxypiperidin-1-yl)-2- | 530 (MNa+) |
| | methylpropanoate | |
| 74 | (S)-4-(methylsulfonyl)benzyl 3-((3R,4S)-4-(4-amino-5- | 568 (MH+) |
| | chloro-2-methoxybenzamido)-3-methoxypiperidin-1-yl)-2- | 590 (MNa+) |
| | methylpropanoate | 566 (MH−) |
| 75 | (S)-3-((3R,4S)-4-(4-amino-5-chloro-2- | 400 (MH+) |
| | methoxybenzamido)-3-methoxypiperidin-1-yl)-2- | 438 (MK+) |
| | methylpropanoic acid | 398 (MH−) |
| 76 | (S)-methyl 3-((3R,4S)-4-(4-amino-5-chloro-2- | 414 (MH+) |
| | methoxybenzamido)-3-methoxypiperidin-1-yl)-2- | 436 (MNa+) |
| | methylpropanoate | 412 (MH−) |
| 77 | 4-((3R,4S)-4-(4-amino-5-chloro-2-methoxybenzamido)-3- | 400 (MH+) |
| | methoxypiperidin-1-yl)butanoic acid | 422 (MNa+) |
| 78 | 4-fluorobenzyl 6-((3S,4R)-4-(4-amino-5-chloro-2- | 536 (MH+) |
| | methoxybenzamido)-3-methoxypiperidin-1-yl)hexanoate | |
| 79 | 2-methoxyethyl 6-((3S,4R)-4-(4-amino-5-chloro-2- | 486 (MH+) |
| | methoxybenzamido)-3-methoxypiperidin-1-yl)hexanoate | 508 (MNa+) |
| 80 | neopentyl 6-((3S,4R)-4-(4-amino-5-chloro-2- | 498 (MH+) |
| | methoxybenzamido)-3-methoxypiperidin-1-yl)hexanoate | 520 (MNa+) |
| 81 | pyridin-2-ylmethyl 6-((3S,4R)-4-(4-amino-5-chloro-2- | 519 (MH+) |
| | methoxybenzamido)-3-methoxypiperidin-1-yl)hexanoate | |
| 82 | 2-(piperazin-1-yl)ethyl 6-((3S,4R)-4-(4-amino-5-chloro-2- | 540 (MH+) |
| | methoxybenzamido)-3-methoxypiperidin-1-yl)hexanoate | |
| | hydrochloride | |
| 83 | 2-(dimethylamino)ethyl 6-((3S,4R)-4-(4-amino-5-chloro- | 499 (MH+) |
| | 2-methoxybenzamido)-3-methoxypiperidin-1-yl)hexanoate | |
| 84 | 1-adamantylmethyl 6-{(3S,4R)-4-[(4-amino-5-chloro-2- | 577 (MH+) |
| | methoxybenzoyl)amino]-3-methoxypiperidin-1-yl}hexanoate | |
| 85 | cyclohexyl 6-((3S,4R)-4-(4-amino-5-chloro-2- | 510 (MH+) |
| | methoxybenzamido)-3-methoxypiperidin-1-yl)hexanoate | |
| 86 | 2-adamantyl 6-{(3S,4R)-4-[(4-amino-5-chloro-2- | 562 (MH+) |
| | methoxybenzoyl)amino]-3-methoxypiperidin-1-yl}hexanoate | |
| 87 | bicyclo[2.2.1]heptan-2-yl 6-((3S,4R)-4-(4-amino-5- | 522 (MH+) |
| | chloro-2-methoxybenzamido)-3-methoxypiperidin-1- | |
| | yl)hexanoate | |
| 88 | 2-(2-((3S,4R)-4-(4-amino-5-chloro-2- | 416 (MH+) |
| | methoxybenzamido)-3-methoxypiperidin-1-yl)ethoxy)acetic | 438 (MNa+) |
| | acid | |
| 89 | methyl 2-(2-((3R,4S)-4-(4-amino-5-chloro-2- | 429 (MH+) |
| | methoxybenzamido)-3-methoxypiperidin-1-yl)ethoxy)acetate | 452 (MNa+) |
| 90 | cyclohexyl 2-(2-((3S,4R)-4-(4-amino-5-chloro-2- | 498 (MH+) |
| | methoxybenzamido)-3-methoxypiperidin-1-yl)ethoxy)acetate | 520 (MNa+) |
| 91 | cyclohexyl 2-(2-((3R,4S)-4-(4-amino-5-chloro-2- | 498 (MH+) |
| | methoxybenzamido)-3-methoxypiperidin-1-yl)ethoxy)acetate | 520 (MNa+) |
| 92 | piperidin-4-yl 2-(2-((3R,4S)-4-(4-amino-5-chloro-2- | 535.46 |
| | methoxybenzamido)-3-methoxypiperidin-1-yl)ethoxy)acetate | (calculated) |
| | hydrochloride | |
| 93 | 2-hydroxyethyl 4-(2-((3S,4R)-4-(4-amino-5-chloro-2- | 535 (MH+) |
| | methoxybenzamido)-3-methoxypiperidin-1- | 533 (MH−) |
| | yl)acetamido)benzoate | |
| 94 | 2-amino-2-oxoethyl 4-(2-((3S,4R)-4-(4-amino-5-chloro-2- | 548 (MH+) |
| | methoxybenzamido)-3-methoxypiperidin-1- | 570 (MNa+) |
| | yl)acetamido)benzoate | 546 (MH−) |
| 95 | 2-(piperazin-1-yl)ethyl 4-(2-((3S,4R)-4-(4-amino-5- | 603 (MH+) |
| | chloro-2-methoxybenzamido)-3-methoxypiperidin-1- | |
| | yl)acetamido)benzoate | |
| 96 | 1-(2-((3S,4R)-4-(4-amino-5-chloro-2- | 483 (MH+) |
| | methoxybenzamido)-3-methoxypiperidin-1-yl)acetyl)piperidine- | |
| | 4-carboxylic acid | |
| 97 | methyl 1-(2-((3S,4R)-4-(4-amino-5-chloro-2- | 497 (MH+) |
| | methoxybenzamido)-3-methoxypiperidin-1-yl)acetyl)piperidine- | 519 (MNa+) |
| | 4-carboxylate | |
| 98 | ethyl 1-(2-((3S,4R)-4-(4-amino-5-chloro-2- | 511 (MH+) |
| | methoxybenzamido)-3-methoxypiperidin-1-yl)acetyl)piperidine- | 533 (MNa+) |
| | 4-carboxylate | |
| 99 | 2-methoxyethyl 1-(2-((3S,4R)-4-(4-amino-5-chloro-2- | 541 (MH+) |
| | methoxybenzamido)-3-methoxypiperidin-1-yl)acetyl)piperidine- | 563 (MNa+) |
| | 4-carboxylate | |

Formulation, Administration, and Uses

Dosage rates and routes of administration of the disclosed compounds are similar to those already used in the art and known to the skilled artisan (see, for example, Physicians' Desk Reference, 54th Ed., Medical Economics Company, Montvale, N.J., 2000).

The magnitude of a prophylactic or therapeutic dose of structural and/or functional analog of cisapride in the acute or chronic management of diseases and/or disorders described herein will vary with the severity of the condition to be treated, and the route of administration. The dose, and perhaps the dose frequency, will also vary according to the age, body weight, and response of the individual patient. In general, the total daily dose range for structural and/or functional analogs of cisapride, for the conditions described herein, is from about 1 mg to about 200 mg, in single or divided doses. Preferably, a daily dose range should be between about 5 mg to about 100 mg, in single or divided doses, while most preferably, a daily dose range should be between about 5 mg to about 75 mg, in single or divided doses. It is preferred that the doses are administered from 1 to 4 times a day. In managing the patient, the therapy should be initiated at a lower dose, perhaps about 5 mg to about 10 mg, and increased up to about 50 mg or higher depending on the patient's global response. It is further recommended that children, and patients over 65 years, and those with impaired renal or hepatic function, initially receive low doses, and that they be titrated based on individual response(s) and blood level(s). It may be necessary to use dosages outside these ranges in some cases as will be apparent to those skilled in the art. Further, it is noted that the clinician or treating physician will know how and when to interrupt, adjust, or terminate therapy in conjunction with individual patient response.

The compounds of the subject invention can be formulated according to known methods for preparing pharmaceutically useful compositions. Formulations are described in detail in a number of sources which are well known and readily available to those skilled in the art. For example, Remington's Pharmaceutical Science by E.W. Martin describes formulations which can be used in connection with the subject invention. In general, the compositions of the subject invention are formulated such that an effective amount of the bioactive compound(s) is combined with a suitable carrier in order to facilitate effective administration of the composition.

The compositions of the subject invention include compositions such as suspensions, solutions and elixirs; aerosols; or carriers such as starches, sugars, microcrystalline cellulose, diluents, granulating agents, lubricants, binders, disintegrating agents, and the like, in the case of oral solid preparations (such as powders, capsules, and tablets) with the oral solid preparations being preferred over the oral liquid preparations. A preferred oral solid preparation is capsules. The most preferred oral solid preparation is tablets. Preferred amounts of active ingredient (i.e., an structural and/or functional analog of cisapride) in a solid dosage form are about 5 mg, 10 mg, and 25 mg.

Further, acceptable carriers can be either solid or liquid. Solid form preparations include powders, tablets, pills, capsules, cachets, suppositories and dispersible granules. A solid carrier can be one or more substances which may act as diluents, flavoring agents, solubilizers, lubricants, suspending agents, binders, preservatives, tablet disintegrating agents or encapsulating materials.

The disclosed pharmaceutical compositions may be subdivided into unit doses containing appropriate quantities of the active component. The unit dosage form can be a packaged preparation, such as packeted tablets, capsules, and powders in paper or plastic containers or in vials or ampules. Also, the unit dosage can be a liquid based preparation or formulated to be incorporated into solid food products, chewing gum, or lozenge.

In addition to the common dosage forms set out above, the compounds of the present invention may also be administered by controlled release means and/or delivery devices such as those described in U.S. Pat. Nos. 3,845,770; 3,916,899; 3,536,809; 3,598,123; and 4,008,719, the disclosures of which are hereby incorporated by reference in their entirety.

Any suitable route of administration may be employed for providing the patient with an effective dosage of a structural and/or functional analog of cisapride. For example, oral, rectal, parenteral (subcutaneous, intramuscular, intravenous), transdermal, and like forms of administration may be employed. Dosage forms include tablets, troches, dispersions, suspensions, solutions, capsules, patches, and the like.

One aspect of the invention provides a method of treating gastroesophageal reflux disease in a mammal, while substantially reducing the concomitant adverse effects associated with the administration of cisapride, which comprises administering to a human in need of such treatment, a therapeutically effective amount of a structural and/or functional analog of cisapride, or a pharmaceutically acceptable salt thereof. A preferred aspect is the treatment of gastroesophageal reflux disease in humans.

Another aspect of the invention provides a composition for the treatment of a human suffering from gastroesophageal reflux disease, which comprises a therapeutically effective amount of a structural and/or functional analog of cisapride, or a pharmaceutically acceptable salt thereof.

Yet another aspect of the present invention provides a method of eliciting an anti-emetic effect in a mammal, while substantially reducing the adverse effects associated with the administration of cisapride, which comprises administering to a mammal in need of such anti-emetic therapy, a therapeutically effective amount of structural and/or functional analogs of cisapride, or a pharmaceutically acceptable salt thereof. Preferably, the mammal is a human.

In an additional aspect, the present invention encompasses an anti-emetic composition for the treatment of a mammal in need of anti-emetic therapy, which comprises a therapeutically effective amount of a structural and/or functional analog of cisapride, or a pharmaceutically acceptable salt thereof.

A further aspect of the present invention includes a method of treating a condition caused by gastrointestinal motility dysfunction in a mammal which comprises administering to a mammal in need of treatment for gastrointestinal motility dysfunction, a therapeutically effective amount of a structural and/or functional analog of cisapride, or a pharmaceutically acceptable salt thereof. Conditions caused by gastrointestinal motility dysfunction include, but are not limited to, dyspepsia, gastroparesis, constipation, post-operative ileus, and intestinal pseudo-obstruction. Preferably, the mammal is a human.

The observation that cisapride enters the central nervous system and binds to 5HT₄ receptors indicates that cisapride may have centrally-mediated effects. Cisapride is a potent ligand at 5HT₄ receptors, and these receptors are located in several areas of the central nervous system. Modulation of serotonergic systems has a variety of behavioral effects. Accordingly, the compounds of the subject invention can be used in the treatment of: 1) cognitive disorders, including but not limited to Alzheimer's disease; 2) behavioral disorders, including but not limited to schizophrenia, mania, obsessive-compulsive disorder, and psychoactive substance use disorders; 3) mood disorders, including but not limited to depression and anxiety; and 4) disorders of control of autonomic function, including but not limited to essential hypertension and sleep disorders.

Accordingly, the present invention also provides methods of treating cognitive, behavioral, mood, or autonomic function control disorders in a mammal comprising the administration of a therapeutically effective amount of structural and/or functional analog of cisapride, or a pharmaceutically acceptable salt thereof. Preferably, the mammal is a human.

THE Compounds Bind to 5-HT₄ Receptors

The 5-HT₄ receptor is known to be the major receptor subtype involved in the prokinetic activity of cisapride in the gut. The compounds of the invention interact with the 5-HT₄ receptor, as shown in Table 1.

Table 1 shows the relative affinity to 5-HT₄ for compounds of the presents invention.

5-HT₄ Receptor Binding - Guinea Pig Striatum

| Kᵢ (nM) | Compound No. |
|---|---|
| <50 | 93, 94, 95, 10, 11, 25, 34, 46 |
| | 47, 48, 49, 59, 60, 67, 68, 69 |
| 50-500 | 12, 24 |
| >500 | 23, 33, 45, 66 |

5-HT₄ receptor prototypic reference antagonist [³H]GR113808 (0.70 nM)

Affinity for the Cardiac Channel, I_{Kr}

The rapidly activating delayed rectifier potassium (K+) current in humans (human I_{Kr}) is a K+ channel that is encoded by the human-ether-a-go-go-related gene (hERG). Cisapride is known to produce QT interval prolongations via a blockade of I_{Kr}, and it was therefore of interest to determine whether compounds of the invention have important inhibitory effects on human I_{Kr}. The test system was mammalian HEK-293 cells expressing the hERG K+ channels, in which the potassium current was measured by whole cell patch-clamp technique. Overall, the findings indicate that compounds of the invention have a lower pro-arrhythmic potential than cisapride and suggest that they have modest to negligible affinity for human I_{Kr} channels.

TABLE 3
Inhibition of I_{Kr} Activity

| | | Activity of I_{Kr} in HEK Cells |
|---|---|---|
| IC₅₀ | | Compound |
| >25,000 | nM | 93, 25, 33, 47, 10, 45, 66 |
| 10,000-25,000 | nM | 48 |
| 5,000-10,000 | nM | 94 |

Data are normalized to % control tail IKr (current elicited without drug or vehicle present)

Metabolism in Human Microsomal Preparations

The metabolism of these compounds was studied in pooled human microsomes in the presence and absence of the Cytochrome P-450 cofactor NADPH and both the disappearance of parent and the appearance of the corresponding acid metabolite, i.e., the corresponding compound-II isomer, monitored with time.

Metabolism in Fresh Human Blood

Test compounds were dissolved in DMSO to make 12.5 mM stock solution and diluted with water to a final concentration of 2.5 mM (DMSO/H2O=20/80). Fresh blood was collected into heparinized tubes from 3 human donors and blood was stored on ice until incubation. Separate aliquots of blood from each donor were pipetted into 1.5 mL centrifuge tubes and the tubes were pre-incubated in a shaking water bath at 37° C. for 5 minutes. The reaction was initiated by the addition of 10 μL, of the appropriate test compound stock to each tube (final concentration=100 μM). Incubations were quenched after 0, 5, 15, 30 and 60 minutes, by the addition of acetonitrile (750 mL), centrifuged at 12,000 rpm for 2 minutes and the supernatant analyzed on an Agilent 1100 HPLC system. Separations were accomplished on a Keystone Intersil ODS2, 250X4.6 mm, 5 m column. The aqueous mobile phase consisted of 20 mM ammonium acetate buffer (pH 5.7) and the organic phase acetonitrile. A gradient was used: initial condition consisted of 20% acetonitrile for 1 minute. The acetonitrile concentration was increased linearly to 90% over the next 8 minutes and held there for 1 minute. The system was then recycled to initial conditions over the course of 1 minute and held there for 4 minutes before the next injection. The peak area for the parent peak was determined by monitoring absorbance at 240, 254 and 290 nM. The results were expressed as amount of initial compound remaining and data subjected to kinetic analysis using. It should be understood that the examples and aspects described herein are for illustrative purposes only and that various modifications or changes in light thereof will be suggested to persons skilled in the art and are to be included within the spirit and purview of this application and the scope of the appended claims. Further, all patents, patent applications, provisional applications, and publications referred to or cited herein are incorporated by reference in their entirety to the extent they are not inconsistent with the explicit teachings of this specification.

The invention and the manner and process of making and using it, are now described in such full, clear, concise and exact terms as to enable any person skilled in the art to which it pertains, to make and use the same. It is to be understood that the foregoing describes preferred aspects of the invention and that modifications may be made therein without departing from the spirit or scope of the invention as set forth in the claims. To particularly point out and distinctly claim the subject matter regarded as invention, the following claims conclude this specification.

What is claimed is:

1. A compound of the formula: 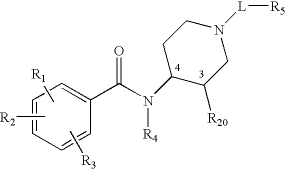
or pharmaceutically acceptable salts thereof, wherein
L is —(C₁-C₄ alkyl)-NR₉—(C₁-C₄ allyl)-, —(C₁-C₄ alkyl)-C(O)NR₉—, —(C₁-C₄ alkyl)-, —(C₁-C₄ alkyl)-NR₉C(O)—, or —C(O)NR₉—(C₁-C₄ alkyl)-;
R₁ is C₁-C₄ alkyl, C₁-C₄ alkoxy, or OH;
R₂ is amino or mono or di(C₁-C₄ alkyl)amino;
R₃ is halogen;
R₄ is H or C₁-C₄ alkyl;
R₅ is phenyl or naphthyl, each of which is substituted with 1 or 2 groups that are independently C₁-C₄ alkyl, C₁-C₄ alkoxy, OH, —O—C₂-C₄ alkanoyl, halogen, halo C₁-C₄ alkyl, halo C₁-C₄ alkoxy, —CO₂R₁₀, —(C₁-C₄ alkyl)-CO₂R₁₀;
R₉ is H or C₁-C₄ alkyl;
R₁₀ at each occurrence is independently H, C₁-C₄ alkyl optionally substituted with one group that is selected from a 5 or 6 membered monocyclic heterocycloalkyl ring, and OH, quinuclidinyl, —C(O)NH₂, —C(O)NH(C₁-C₄ alkyl), —C(O)NH(C₁-C₄ alkyl) or piperidinyl optionally substituted with C₁-C₄ alkyl; and
R₂₀ is C₁-C₄ alkyl, or C₁-C₄ alkoxy.

2. A compound according to claim 1, wherein
R₁ is chloro.

3. A compound according to claim 1, wherein
R₂ is amino.

4. A compound according to claim 1, wherein
R₃ is methoxy.

5. A compound according to claim 1, wherein
R₄ is H.

6. A compound according to claim 1, wherein
R₁ is chloro; R₂ is amino; R₃ is methoxy; R₄ is H, and R₁, R₂, and R₃ have the following orientation on the phenyl ring:

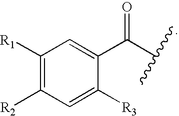

7. A Compound according to claim 6, wherein
L is —(C₁-C₄ alkyl)-NR₉—(C₁-C₄ alkyl)-, —(C₁-C₄ alkyl)-C(O)NR₉—, or —(C₁-C₄ alkyl)-.

8. A compound according to claim 7, having the formula: 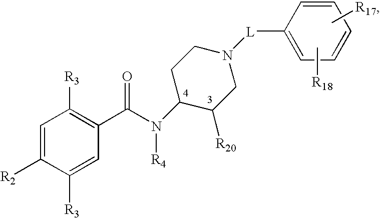
where
R₁₇ is C₁-C₄ alkyl, C₁-C₄ alkoxy, OH, —O—C₂-C₄ alkanoyl, halogen, halo C₁-C₄ alkyl, halo C₁-C₄ alkoxy, —CO₂R₁₀, —(C₁-C₄ alkyl)-CO₂R₁₀; and
R₁₈ is H C₁-C₄ alkyl, C₁-C₄ alkoxy, OH, —O—C₂-C₄ alkanoyl, halogen, halo C₁-C₄ alkyl, halo C₁-C₄ alkoxy, —CO₂R₁₀, —(C₁-C₄ alkyl)-CO₂R₁₀.

9. A compound according to claim 8, wherein
the bonds at positions 3 and 4 of the piperidinyl ring are cis to each other.

10. A compound according to claim 9, wherein
R₁₇ is attached at the para position, relative to the point of attachment of the L group.

11. A compound according to claim 10, wherein
L is —(C₁-C₃ alkyl)-NR₉—(C₁-C₃ alkyl)-;
R₁₀ is H, C₁-C₄ alkyl optionally substituted with one group that is selected from a 5 or 6 membered monocyclic heterocycloalkyl ring, and OH, quinuclidinyl, —C(O)NH₂, or piperidinyl optionally substituted with C₁-C₃ alkyl;
R₁₇ is OH, —O—C₂-C₄ alkanoyl, —CO₂R₁₀, or —(C₁-C₄ alkyl)-CO₂R₁₀; and
R₁₈ is H.

12. A compound according to claim 11, having the formula:

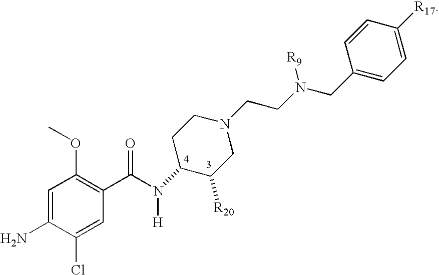

13. A compound according to claim 10, wherein
L, is —(C₁-C₃ alkyl)-C(O)NR₉—;
R₁₀ is H, C₁-C₄ alkyl optionally substituted with one group that is selected from a 5 or 6 membered monocyclic heterocycloalkyl ring, and OH, quinuclidinyl, —C(O)NH₂, or piperidinyl optionally substituted with C₁-C₃ alkyl;
R₁₇ is OH, —O—C₂-C₄ alkanoyl, —CO₂R₁₀, or —(C₁-C₄ alkyl)-CO₂R₁₀; and
R₁₈ is H.

14. A compound according to claim 13, having the formula:

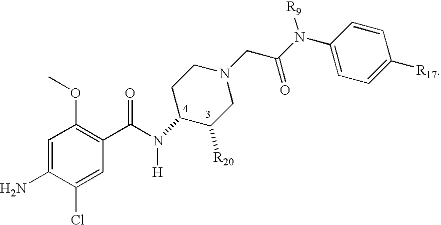

15. A compound according to claim 10, wherein
L is —(C₁-C₃ alkyl)-;
R₁₀ is H, C₁-C₄ alkyl optionally substituted with one group that is selected from a 5 or 6 membered monocyclic heterocycloalkyl ring, and OH, quinuclidinyl, —C(O)NH₂, or piperidinyl optionally substituted with C₁-C₃ alkyl;
R₁₇ is OH, —O—C₂-C₄ alkanoyl, —CO₂R₁₀ or —(C₁-C₄ alkyl)-CO₂R₁₀; and
R₁₈ is H.

16. A compound according to claim 15, having the formula:

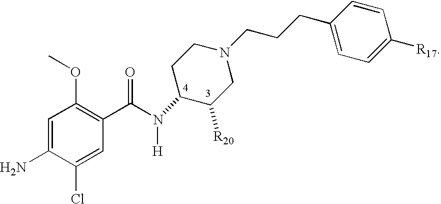

17. A compound according to claim 10, wherein
L, is —(C₁-C₄ alkyl)-NR₉C(O)—;
R₁₀ is H, C₁-C₄ alkyl optionally substituted with one group that is selected from a 5 or 6 membered monocyclic heterocycloalkyl ring, and OH, quinuclidinyl, —C(O)NH₂, or piperidinyl optionally substituted with C₁-C₃ alkyl;
R₁₇ is OH, —O—C₂-C₄ alkanoyl, —CO₂R₁₀, or —(C₁-C₄ alkyl)-CO₂R₁₀; and
R₁₈ is H.

18. A compound according to claim 17, having the formula:

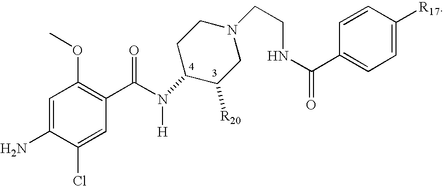

19. A compound according to claim 1, that is
2-(pyrrolidin-1-yl)ethyl 4-(((2-((3S,4R)-4-(4-amino-5-chloro-2-methoxybenzamido)-3-methoxypiperidin-1-yl)ethyl)(methyl)amino)methyl)benzoate;
1-methylpiperidin-4-yl 4-(((2-((3S,4R)-4-(4-amino-5-chloro-2-methoxybenzamido)-3-methoxypiperidin-1-yl)ethyl)(methyl)amino)methyl)benzoate;
2-morpholinoethyl 4-(((2-((3S,4R)-4-(4-amino-5-chloro-2-methoxybenzamido)-3-methoxypiperidin-1-yl)ethyl)(methyl)amino)methyl)benzoate;
4-((2-((3S,4R)-4-(4-amino-5-chloro-2-methoxybenzamido)-3-methoxypiperidin-1-yl)ethylamino)methyl)benzoic acid;
3-hydroxypropyl 4-((2-((3S,4R)-4-(4-amino-5-chloro-2-methoxybenzamido)-3-methoxypiperidin-1-yl)ethylamino)methyl)benzoate;
piperidin-4-yl 4-((2-((3S,4R)-4-(4-amino-5-chloro-2-methoxybenzamido)-3-methoxypiperidin-1-yl)ethylamino)methyl)benzoate;
4-(2-((3S,4R)-4-(4-amino-5-chloro-2-methoxybenzamido)-3-methoxypiperidin-1-yl)ethylcarbamoyl)benzoic acid;
piperidin-4-yl 4-(2-((3S,4R)-4-(4-amino-5-chloro-2-methoxybenzamido)-3-methoxypiperidin-1-yl)ethylcarbamoyl)benzoate;
2-(4-(2-((3S,4R)-4-(4-amino-5-chloro-2-methoxybenzamido)-3-methoxypiperidin-1-yl)acetamido)phenyl)acetic acid;
ethyl 2-(4-(2-((3S,4R)-4-(4-amino-5-chloro-2-methoxybenzamido)-3-methoxypiperidin-1-yl)acetamido)phenyl)acetate;
1-methylpiperidin-4-yl 2-(4-(2-((3S,4R)-4-(4-amino-5-chloro-2-methoxybenzamido)-3-methoxypiperidin-1-yl)acetamido)phenyl)acetate;
3-hydroxypropyl 2-(4-(2-((3S,4R)-4-(4-amino-5-chloro-2-methoxybenzamido)-3-methoxypiperidin-1-yl)acetamido)phenyl)acetate;
quinuclidin-3-yl 2-(4-(2-((3S,4R)-4-(4-amino-5-chloro-2-methoxybenzamido)-3-methoxypiperidin-1-yl)acetamido)phenyl)acetate;
4-amino-5-chloro-N-((3S,4R)-1-(2-(4-hydroxyphenylamino)-2-oxoethyl)-3-methoxypiperidin-4-yl)-2-methoxybenzamide;
4-(2-((3S,4R)-4-(4-amino-5-chloro-2-methoxybenzamido)-3-methoxypiperidin-1-yl)acetamido)phenyl acetate;
4-(3-((3S,4R)-4-(4-amino-5-chloro-2-methoxybenzamido)-3-methoxypiperidin-1-yl)propyl)benzoic acid;
2-morpholinoethyl 4-(3-((3S,4R)-4-(4-amino-5-chloro-2-methoxybenzamido)-3-methoxypiperidin-1-yl)propyl)benzoate;
2-(pyrrolidin-1-yl)ethyl 4-(3-((3S,4R)-4-(4 amino-5-chloro-2-methoxybenzamido)-3-methoxypiperidin-1-yl)propyl)benzoate;
1-methylpiperidin-4-yl 4-(3-((3S,4R)-4-(4-amino-5-chloro-2-methoxybenzamido)-3-methoxypiperidin-1-yl)propyl)benzoate;
2-hydroxyethyl 4-(2-((3S,4R)-4-(4-amino-5-chloro-2-methoxybenzamido)-3-methoxypiperidin-1-yl)acetamido)benzoate;
2-amino-2-oxoethyl 4-(2-((3S,4R)-4-(4-amino-5-chloro-2-methoxybenzamido) 3-methoxypiperidin-1-yl)acetamido)benzoate;
2-(piperazin-1-yl)ethyl 4-(2-((3S,4R)-4-(4-amino-5-chloro-2-methoxybenzamido)-3-methoxypiperidin-1-yl)acetamido)benzoate; or pharmaceutically acceptable salts thereof.

20. A composition comprising a compound or pharmaceutically acceptable salt of claim 1 and at least one pharmaceutically acceptable carrier, solvent, adjuvant, or excipient.

21. A method of treating emesis, dyspepsia, gastroparesis, constipation, intestinal pseudo-obstruction, gastroesophageal reflux, or post-operative ileus, the method comprising administering a compound or salt according to claim 1 to a patient in need of such treatment.

22. A method according to claim 21, wherein the compound or salt is administered intra venously.
